SEQUENCE LISTING (230 sequences)

SEQ ID NO: 1 - DNA, 888 bp, Homo sapiens
atggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctga

SEQ ID NO: 2 - DNA, 888 bp, mus musculus
atggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 3 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 4 - protein, 295 aa, Mus musculus
MEHQLLCCEVETIRRAYPDTNLLNDRVLRAMLKTEETCAPSVSYFKCVQKEIVPSMRKIVATWMLEVCEEQKCEEEVFPLAMNYLDRFLSLEPLKKSRLQLLGATCMFVASKMKETIPLTAEKLCIYTDNSIRPEELLQMELLLVNKLKWNLAAMTPHDFIEHFLSKMPEADENKQTIRKHAQTFVALCATDVKFISNPPSMVAAGSVVAAMQGLNLGSPNNFLSCYRTTHFLSRVIKCDPDCLRACQEQIEALLESSLRQAQQNVDPKATEEEGEVEEEAGLACTPTDVRDVDI

SEQ ID NO: 5 - DNA, 897 bp, artificial sequence
  hybrid molecul for screening siRNA
ggccgcgccatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 6 - DNA, 894 bp, artificial sequence
  hybrid molecul for screening siRNA
gccaccatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 7 - DNA, 903 bp, artificial sequence
  hybrid molecul for screening siRNA
ggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctga

SEQ ID NO: 8 - DNA, 903 bp, artificial sequence
  hybrid molecul for screening siRNA
ggaagagcgccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctga

SEQ ID NO: 9 - DNA, 894 bp, artificial sequence
  hybrid molecul for screening siRNA
gccaccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctga

SEQ ID NO: 10 - DNA, 974 bp, artificial sequence
  hybrid molecul for screening siRNA
ggccgcgccatatggactacaaggacgacgatgacaagctcgatggaggatacccctacgacgtgcccgactacgccggaggactcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 11 - DNA, 1052 bp, Artificial sequence
  hybrid molecul for screening siRNA
ggatcccagtgtggtggtacggcggccgcgccatggactacaaggacgacgatgacaagctcgatggaggatacccctacgacgtgcccgactacgccggaggactcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagaattccatatgcttgtggtagttggagctgttggcgtaggcaagagtgccagatct

SEQ ID NO: 12 - DNA, 1052 bp, Artificial sequence
  hybrid molecul for screening siRNA
ggatcccagtgtggtggtacggcggccgcgccatggactacaaggacgacgatgacaagctcgatggaggatacccctacgacgtgcccgactacgccggaggactcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagaattccatatgcttgtggtagttggagctggtggcgtaggcaagagtgccagatct

SEQ ID NO: 13 - DNA, 889 bp, Artificial sequence
  hybrid molecul for screening siRNA
ggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagcggccgcgactctagatcataatcagccataccacatttgtagaggttttacttgctttaaaaaacctcccacacctccccctgaacctgaaacataaaatgaatgcaattccatatgcttgtggtagttggagctgttggcgtaggcaagagtgccaagatct

SEQ ID NO: 14 - DNA, 832 bp, Artificial sequence
  hybrid molecul for screening siRNA
ggatccgccaccatggactacaaggacgacgatgacaaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtacccctacgacgtgcccgactacgcctaggaattccatatgcttgtggtagttggagctgttggcgtaggcaagagtgccaagatct

SEQ ID NO: 15 - DNA, 967 bp, Artificial sequence
  hybrid molecul for screening siRNA
ggatccggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagaattccatatgcttgtggtagttggagctgttggcgtaggcaagagtgccaagatct

SEQ ID NO: 16 - DNA, 719 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattgtagttagtttagaccggttgattttggtctagctacagtgaaatctcgatggagtgggtacgcgtagatct

SEQ ID NO: 17 - DNA, 719 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattgtagttagtttagaccggttgattttggtctagctacagagaaatctcgatggagtgggtacgcgtagatct

SEQ ID NO: 18 - DNA, 1058 bp, Artificial sequence
  hybrid molecul for screening siRNA
ggatccacgcgtgccaccatgctcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagaattctacccctacgacgtgcccgactacgcctaacgtgacacgttcggagaattacatatgagatcccaattgtagttagtttagaccggttgattttggtctagctacagtgaaatctcgatggagtgggtacgcgtagatct

SEQ ID NO: 19 - DNA, 1058 bp, Artificial sequence
  hybrid molecul for screening siRNA
ggatccacgcgtgccaccatgctcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagaattctacccctacgacgtgcccgactacgcctaacgtgacacgttcggagaattacatatgagatcccaattgtagttagtttagaccggttgattttggtctagctacagagaaatctcgatggagtgggtacgcgtagatct

SEQ ID NO: 20 - DNA, 657 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattccatatgcttgtggtagttggagctgttggcgtaggcaagagtgccagatctcgaccctgtggaatgtgtgtcagttag

SEQ ID NO: 21 - DNA, 699 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattccatatgcttgtggtagttggagctgttggcgtaggcaagagtgccagatct

SEQ ID NO: 22 - DNA, 719 bp, Artificial sequence; identical to SEQ ID NO: 17
  hybrid molecul for screening siRNA

SEQ ID NO: 23 - DNA, 713 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattgcacgtgactagtacttgtggtagttggagctggtggcgtaggcaagagtgcctcacgtgagatct

SEQ ID NO: 24 - DNA, 713 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattgcacgtgactagtacttgtggtagttggagctgttggcgtaggcaagagtgcctcacgtgagatct

SEQ ID NO: 25 - DNA, 660 bp, Artificial sequence
  hybrid molecul for screening siRNA
ggatccgccaccatgggcaaaccgattccgaacccgctgctgggcctggatagcaccctcgaggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctacccctacgacgtgcccgactacgcctaacgtgacacgttcggagaattacatatgagatcccaattgcacgtgagatct

SEQ ID NO: 26 - RNA, 888 nt, Homo sapiens
auggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucuga

SEQ ID NO: 27 - RNA, 888 nt, Mus musculus
auggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucuga

SEQ ID NO: 28 - DNA, 492 bp, Artificial Sequence
  Internal ribosome entry site
ccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaacccccaggcccacgttggagttggatagttgggaaagagtcaaatggctttcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatacc

SEQ ID NO: 29 - RNA, 492 nt, Artificial Sequence
  Internal ribosome entry site
ccgcccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaacccccaggcccacguuggaguuggauaguugggaaagagucaaauggcuuuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgauaauacc

SEQ ID NO: 30 - RNA, 897 nt, Artificial Sequence
  region 1 of the nucleic acid according to the invention
ggccgcgccauggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucuga

SEQ ID NO: 31 - RNA, 894 nt, Artificial Sequence
  region 1 of the nucleic acid according to the invention
gccaccauggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucuga

SEQ ID NO: 32 - RNA, 903 nt, Artificial Sequence
  region 1 of the nucleic acid according to the invention
ggaagagccccagccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucuga

SEQ ID NO: 33 - RNA, 903 nt, Artificial Sequence
  region 1 of the nucleic acid according to the invention
ggaagagcgccagccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucuga

SEQ ID NO: 34 - RNA, 894 nt, Artificial Sequence
  region 1 of the nucleic acid according to the invention
gccaccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucuga

SEQ ID NO: 35 - RNA, 974 nt, Artificial Sequence
  region 1 of the nucleic acid according to the invention
ggccgcgccauauggacuacaaggacgacgaugacaagcucgauggaggauaccccuacgacgugcccgacuacgccggaggacucgaggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucuga

SEQ ID NO: 36 - RNA, 1052 nt, Artificial sequence
  hybrid molecule according to the invention
ggaucccaguguggugguacggcggccgcgccauggacuacaaggacgacgaugacaagcucgauggaggauaccccuacgacgugcccgacuacgccggaggacucgaggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucugagaauuccauaugcuugugguaguuggagcuguuggcguaggcaagagugccagaucu

SEQ ID NO: 37 - RNA, 1052 nt, Artificial sequence
  hybrid molecule according to the invention
ggaucccaguguggugguacggcggccgcgccauggacuacaaggacgacgaugacaagcucgauggaggauaccccuacgacgugcccgacuacgccggaggacucgaggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucugagaauuccauaugcuugugguaguuggagcugguggcguaggcaagagugccagaucu

SEQ ID NO: 38 - RNA, 889 nt, Artificial sequence
  hybrid molecule according to the invention
ggauccgccaccauggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaaguaaagcggccgcgacucuagaucauaaucagccauaccacauuuguagagguuuuacuugcuuuaaaaaaccucccacaccucccccugaaccugaaacauaaaaugaaugcaauuccauaugcuugugguaguuggagcuguuggcguaggcaagagugccaagaucu

SEQ ID NO: 39 - RNA, 832 nt, Artificial sequence
  hybrid molecule according to the invention
ggauccgccaccauggacuacaaggacgacgaugacaaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaaguaccccuacgacgugcccgacuacgccuaggaauuccauaugcuugugguaguuggagcuguuggcguaggcaagagugccaagaucu

SEQ ID NO: 40 - RNA, 967 nt, Artificial sequence
  hybrid molecule according to the invention
ggauccggaagagccccagccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucugagaauuccauaugcuugugguaguuggagcuguuggcguaggcaagagugccaagaucu

SEQ ID NO: 41 - RNA, 719 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauuguaguuaguuuagaccgguugauuuuggucuagcuacagugaaaucucgauggaguggguacgcguagaucu

SEQ ID NO: 42 - RNA, 719 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauuguaguuaguuuagaccgguugauuuuggucuagcuacagagaaaucucgauggaguggguacgcguagaucu

SEQ ID NO: 43 - RNA, 1058 nt, Artificial sequence
  hybrid molecule according to the invention
ggauccacgcgugccaccaugcucgaggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucugagaauucuaccccuacgacgugcccgacuacgccuaacgugacacguucggagaauuacauaugagaucccaauuguaguuaguuuagaccgguugauuuuggucuagcuacagugaaaucucgauggaguggguacgcguagaucu

SEQ ID NO: 44 - RNA, 1058 nt, Artificial sequence
  hybrid molecule according to the invention
ggauccacgcgugccaccaugcucgaggaacaccagcuccugugcugcgaaguggagaccauccgccgcgcguacccugacaccaaucuccucaacgaccgggugcugcgagccaugcucaagacggaggagaccugugcgcccuccguaucuuacuucaagugcgugcagaaggagauugugccauccaugcggaaaaucguggccaccuggaugcuggaggucugugaggagcagaagugcgaagaggaggucuucccgcuggccaugaacuaccuggaccgcuuccugucccuggagcccuugaagaagagccgccugcagcugcugggggccaccugcauguucguggccucuaagaugaaggagaccauucccuugacugccgagaaguugugcaucuacacugacaacucuauccggcccgaggagcugcugcaaauggaacugcuucuggugaacaagcucaaguggaaccuggccgccaugacuccccacgauuucaucgaacacuuccucuccaaaaugccagaggcggaugagaacaagcagaccauccgcaagcaugcacagaccuuuguggcccucugugccacagaugugaaguucauuuccaacccacccuccaugguagcugcugggagcgugguggcugcgaugcaaggccugaaccugggcagccccaacaacuuccucuccugcuaccgcacaacgcacuuucuuuccagagucaucaagugugacccggacugccuccgugccugccaggaacagauugaagcccuucuggagucaagccugcgccaggcccagcagaacgucgaccccaaggccacugaggaggagggggaaguggaggaagaggcuggucuggccugcacgcccaccgacgugcgagauguggacaucugagaauucuaccccuacgacgugcccgacuacgccuaacgugacacguucggagaauuacauaugagaucccaauuguaguuaguuuagaccgguugauuuuggucuagcuacagagaaaucucgauggaguggguacgcguagaucu

SEQ ID NO: 45 - RNA, 657 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauuccauaugcuugugguaguuggagcuguuggcguaggcaagagugccagaucucgacccuguggaaugugugucaguuag

SEQ ID NO: 46 - RNA, 699 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauuccauaugcuugugguaguuggagcuguuggcguaggcaagagugccagaucu

SEQ ID NO: 47 - RNA, 719 nt, Artificial sequence; identical to SEQ ID NO: 42
  hybrid molecule according to the invention

SEQ ID NO: 48 - RNA, 713 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauugcacgugacuaguacuugugguaguuggagcugguggcguaggcaagagugccucacgugagaucu

SEQ ID NO: 49 - RNA, 713 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauugcacgugacuaguacuugugguaguuggagcuguuggcguaggcaagagugccucacgugagaucu

SEQ ID NO: 50 - RNA, 660 nt, Artificial sequence
  hybrid molecule according to the invention
ggauccgccaccaugggcaaaccgauuccgaacccgcugcugggccuggauagcacccucgaggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggaauucuaccccuacgacgugcccgacuacgccuaacgugacacguucggagaauuacauaugagaucccaauugcacgugagaucu

SEQ ID NO: 51 - protein, 14 aa, Artificial Sequence
  peptide from 95 to 108 of V5 tag
GKPIPNPLLGLDST

SEQ ID NO: 52 - DNA, 27 bp, Artificial Sequence
  Forward primer
ggtctggcctgcgcgcccaccgacgtg

SEQ ID NO: 53 - DNA, 27 bp, Artificial Sequence
  reverse primer
cacgtcggtgggcgcgcaggccagacc

SEQ ID NO: 54 - DNA, 24 bp, Artificial Sequence
  primer forward
tatgctatccagaaaacccctcaa

SEQ ID NO: 55 - DNA, 20 bp, Artificial Sequence
  primer forward
ggagcgagaccccactaaca

SEQ ID NO: 56 - DNA, 23 bp, Artificial Sequence
  primer forward
agaccttgcactctttggacatg

SEQ ID NO: 57 - DNA, 20 bp, Artificial Sequence
  primer forward
aggtgctgggagctgctaca

SEQ ID NO: 58 - DNA, 20 bp, Artificial Sequence
  primer forward
atcgagtccggtagccggtg

SEQ ID NO: 59 - DNA, 28 bp, Artificial Sequence
  primer forward
cttagtgaacttctgttgtcctccagca

SEQ ID NO: 60 - DNA, 20 bp, Artificial Sequence
  primer forward
aggagcagaagtgcgaagag

SEQ ID NO: 61 - DNA, 20 bp, Artificial Sequence
  primer forward
cctgtcccctcagttcatgt

SEQ ID NO: 62 - DNA, 22 bp, Artificial Sequence
  primer reverse
gtatgttcggcttcccattctc

SEQ ID NO: 63 - DNA, 20 bp, Artificial Sequence
  primer reverse
acatactcagcaccggcctc

SEQ ID NO: 64 - DNA, 18 bp, Artificial Sequence
  primer reverse
gccaacggagcaggttga

SEQ ID NO: 65 - DNA, 25 bp, Artificial Sequence
  primer reverse
ggtgtgtatggatcaccagttccta

SEQ ID NO: 66 - DNA, 20 bp, Artificial Sequence
  primer reverse
aaagcgactccagctctgct

SEQ ID NO: 67 - DNA, 19 bp, Artificial Sequence
  primer reverse
gaaacctagccaaaccgcc

SEQ ID NO: 68 - DNA, 28 bp, Artificial Sequence
  primer reverse
aggcaaactgagcaccataatttacaaa

SEQ ID NO: 69 - DNA, 20 bp, Artificial Sequence
  primer reverse
cacaacttctcggcagtcaa

SEQ ID NO: 70 - DNA, 20 bp, Artificial Sequence
  primer reverse
cccatggtcttcttctgcat

SEQ ID NO: 71 - RNA, 21 nt, Artificial sequence
  Active anti-sens
aauucuccgaacgugucacgu

SEQ ID NO: 72 - RNA, 21 nt, Artificial sequence
  Active anti-sens
uagucgggcacgucguagggg

SEQ ID NO: 73 - RNA, 21 nt, Artificial sequence
  Active anti-sens
gucaucgucguccuuguaguc

SEQ ID NO: 74 - RNA, 21 nt, Artificial sequence
  Active anti-sens
cgacuugucaucgucguccuu

SEQ ID NO: 75 - RNA, 21 nt, Artificial sequence
  Active anti-sens
gagcuugucaucgucguccuu

SEQ ID NO: 76 - RNA, 21 nt, Artificial sequence
  Active anti-sens
ccacagaugugaaguucauuu

SEQ ID NO: 77 - RNA, 21 nt, Artificial sequence
  Active anti-sens
aacaccagcuccugugcugcg

SEQ ID NO: 78 - RNA, 21 nt, Artificial sequence
  Active anti-sens
caggaacagauugaagcccuu

SEQ ID NO: 79 - RNA, 21 nt, Artificial sequence
  Active anti-sens
cggguucggaaucgguuugcc

SEQ ID NO: 80 - RNA, 21 nt, Artificial sequence
  Active anti-sens
gcggguucggaaucgguuugc

SEQ ID NO: 81 - RNA, 21 nt, Artificial sequence
  Active anti-sens
cagcggguucggaaucgguuu

SEQ ID NO: 82 - RNA, 21 nt, Artificial sequence
  Active anti-sens
gcagcggguucggaaucgguu

SEQ ID NO: 83 - RNA, 21 nt, Artificial sequence
  Active anti-sens
agcagcggguucggaaucggu

SEQ ID NO: 84 - RNA, 21 nt, Artificial sequence
  Active anti-sens
cagcagcggguucggaaucgg

SEQ ID NO: 85 - RNA, 21 nt, Artificial sequence
  Active anti-sens
ccagcagcggguucggaaucg

SEQ ID NO: 86 - RNA, 21 nt, Artificial sequence
  Active anti-sens
cccagcagcggguucggaauc

SEQ ID NO: 87 - RNA, 21 nt, Artificial sequence
  Active anti-sens
gcccagcagcggguucggaau

SEQ ID NO: 88 - RNA, 21 nt, Artificial sequence
  Active anti-sens
ggcccagcagcggguucggaa

SEQ ID NO: 89 - RNA, 21 nt, Artificial sequence
  Active anti-sens
aggcccagcagcggguucgga

SEQ ID NO: 90 - RNA, 21 nt, Artificial sequence
  Active anti-sens
caggcccagcagcggguucgg

SEQ ID NO: 91 - RNA, 21 nt, Artificial sequence
  Active anti-sens
ccaggcccagcagcggguucg

SEQ ID NO: 92 - RNA, 21 nt, Artificial sequence
  Active anti-sens
uccaggcccagcagcggguuc

SEQ ID NO: 93 - RNA, 21 nt, Artificial sequence
  Active anti-sens
auccaggcccagcagcggguu

SEQ ID NO: 94 - RNA, 21 nt, Artificial sequence
  Active anti-sens
uauccaggcccagcagcgggu

SEQ ID NO: 95 - RNA, 21 nt, Artificial sequence
  Active anti-sens
cuauccaggcccagcagcggg

SEQ ID NO: 96 - RNA, 21 nt, Artificial sequence
  Active anti-sens
gcuauccaggcccagcagcgg

SEQ ID NO: 97 - RNA, 21 nt, Artificial sequence
  Active anti-sens
ugcuauccaggcccagcagcg

SEQ ID NO: 98 - RNA, 21 nt, Artificial sequence
  Active anti-sens
gugcuauccaggcccagcagc

SEQ ID NO: 99 - RNA, 21 nt, Artificial sequence
  Active anti-sens
ggugcuauccaggcccagcag

SEQ ID NO: 100 - RNA, 21 nt, Artificial sequence
  Active anti-sens
acagcuccaacuaccacaagc

SEQ ID NO: 101 - RNA, 21 nt, Artificial sequence
  Active anti-sens
aacagcuccaacuaccacaag

SEQ ID NO: 102 - RNA, 21 nt, Artificial sequence
  Active anti-sens
caacagcuccaacuaccacaa

SEQ ID NO: 103 - RNA, 21 nt, Artificial sequence
  Active anti-sens
ccaacagcuccaacuaccaca

SEQ ID NO: 104 - RNA, 21 nt, Artificial sequence
  Active anti-sens
gccaacagcuccaacuaccac

SEQ ID NO: 105 - RNA, 21 nt, Artificial sequence
  Active anti-sens
cgccaacagcuccaacuacca

SEQ ID NO: 106 - RNA, 21 nt, Artificial sequence
  Active anti-sens
acgccaacagcuccaacuacc

SEQ ID NO: 107 - RNA, 21 nt, Artificial sequence
  Active anti-sens
uacgccaacagcuccaacuac

SEQ ID NO: 108 - RNA, 21 nt, Artificial sequence
  Active anti-sens
cuacgccaacagcuccaacua

SEQ ID NO: 109 - RNA, 21 nt, Artificial sequence
  Active anti-sens
ccuacgccaacagcuccaacu

SEQ ID NO: 110 - RNA, 21 nt, Artificial sequence
  Active anti-sens
gccuacgccaacagcuccaac

SEQ ID NO: 111 - RNA, 21 nt, Artificial sequence
  Active anti-sens
ugccuacgccaacagcuccaa

SEQ ID NO: 112 - RNA, 21 nt, Artificial sequence
  Active anti-sens
uugccuacgccaacagcucca

SEQ ID NO: 113 - RNA, 21 nt, Artificial sequence
  Active anti-sens
cuugccuacgccaacagcucc

SEQ ID NO: 114 - RNA, 21 nt, Artificial sequence
  Active anti-sens
ucuugccuacgccaacagcuc

SEQ ID NO: 115 - RNA, 21 nt, Artificial sequence
  Active anti-sens
cucuugccuacgccaacagcu

SEQ ID NO: 116 - RNA, 21 nt, Artificial sequence
  Active anti-sens
acucuugccuacgccaacagc

SEQ ID NO: 117 - RNA, 21 nt, Artificial sequence
  Active anti-sens
cacucuugccuacgccaacag

SEQ ID NO: 118 - RNA, 21 nt, Artificial sequence
  Active anti-sens
gcacucuugccuacgccaaca

SEQ ID NO: 119 - RNA, 21 nt, Artificial sequence
  Active anti-sens
ggcacucuugccuacgccaac

SEQ ID NO: 120 - RNA, 21 nt, Artificial Sequence
  Active anti-sens
uggcacucuugccuacgccaa

SEQ ID NO: 121 - DNA, 21 bp, Artificial sequence
  Non-active Sense sequence
acgugacacguucggagaatt

SEQ ID NO: 122 - DNA, 21 bp, Artificial sequence
  Non-active Sense sequence
ccuacgacgugcccgacuatt

SEQ ID NO: 123 - DNA, 21 bp, Artificial sequence
  Non-active Sense sequence
cuacaaggacgacgaugactt

SEQ ID NO: 124 - DNA, 21 bp, Artificial sequence
  Non-active Sense sequence
ggacgacgaugacaagucgtt

SEQ ID NO: 125 - DNA, 21 bp, Artificial sequence
  Non-active Sense sequence
ggacgacgaugacaagcuctt

SEQ ID NO: 126 - DNA, 23 bp, Artificial sequence
  Non-active Sense sequence
aaaugaacuucacaucuguggtt

SEQ ID NO: 127 - DNA, 23 bp, Artificial sequence
  Non-active Sense sequence
cgcagcacaggagcugguguutt

SEQ ID NO: 128 - DNA, 23 bp, Artificial sequence
  Non-active Sense sequence
aagggcuucaaucuguuccugtt

SEQ ID NO: 129 - DNA, 21 bp, Artificial sequence
  Non-active Sense sequence
caaaccgauuccgaacccgtt

SEQ ID NO: 130 - DNA, 21 bp, Artificial sequence
  Non-active Sense sequence
aaaccgauuccgaacccgctt

SEQ ID NO: 131 - DNA, 21 bp, Artificial sequence
  Non-active Sense sequence
accgauuccgaacccgcugtt

SEQ ID NO: 132 - DNA, 21 bp, Artificial sequence
  Non-active Sense sequence
ccgauuccgaacccgcugctt

SEQ ID NO: 133 - DNA, 21 bp, Artificial sequence
  Non-active Sense sequence
cgauuccgaacccgcugcutt

SEQ ID NO: 134 - DNA, 21 bp, Artificial sequence
  Non-active Sense sequence
gauuccgaacccgcugcugtt

SEQ ID NO: 135 - DNA, 21 bp, Artificial sequence
  Non-active Sense sequence
auuccgaacccgcugcuggtt

SEQ ID NO: 136 - DNA, 21 bp, Artificial sequence
  Non-active Sense sequence
uuccgaacccgcugcugggtt

SEQ ID NO: 137 - DNA, 21 bp, Artificial sequence
  Non-active Sense sequence
uccgaacccgcugcugggctt

SEQ ID NO: 138 - DNA, 21 bp, Artificial sequence
  Non-active Sense sequence
ccgaacccgcugcugggcctt

SEQ ID NO: 139 - DNA, 21 bp, Artificial sequence
  Non-active Sense sequence
cgaacccgcugcugggccutt

SEQ ID NO: 140 - DNA, 21 bp, Artificial sequence
  Non-active Sense sequence
gaacccgcugcugggccugtt

SEQ ID NO: 141 - DNA, 21 bp, Artificial sequence
  Non-active Sense sequence
aacccgcugcugggccuggtt

SEQ ID NO: 142 - DNA, 21 bp, Artificial sequence
  Non-active Sense sequence
acccgcugcugggccuggatt

SEQ ID NO: 143 - DNA, 21 bp, Artificial sequence
  Non-active Sense sequence
cccgcugcugggccuggautt

SEQ ID NO: 144 - DNA, 21 bp, Artificial sequence
  Non-active Sense sequence
ccgcugcugggccuggauatt

SEQ ID NO: 145 - DNA, 21 bp, Artificial sequence
  Non-active Sense sequence
cgcugcugggccuggauagtt

SEQ ID NO: 146 - DNA, 21 bp, Artificial sequence
  Non-active Sense sequence
gcugcugggccuggauagctt

SEQ ID NO: 147 - DNA, 21 bp, Artificial sequence
  Non-active Sense sequence
cugcugggccuggauagcatt

SEQ ID NO: 148 - DNA, 21 bp, Artificial sequence
  Non-active Sense sequence
ugcugggccuggauagcactt

SEQ ID NO: 149 - DNA, 21 bp, Artificial sequence
  Non-active Sense sequence
gcugggccuggauagcacctt

SEQ ID NO: 150 - DNA, 21 bp, Artificial sequence
  Non-active Sense sequence
uugugguaguuggagcugutt

SEQ ID NO: 151 - DNA, 21 bp, Artificial sequence
  Non-active Sense sequence
ugugguaguuggagcuguutt

SEQ ID NO: 152 - DNA, 21 bp, Artificial sequence
  Non-active Sense sequence
gugguaguuggagcuguugtt

SEQ ID NO: 153 - DNA, 21 bp, Artificial sequence
  Non-active Sense sequence
ugguaguuggagcuguuggtt

SEQ ID NO: 154 - DNA, 21 bp, Artificial sequence
  Non-active Sense sequence
gguaguuggagcuguuggctt

SEQ ID NO: 155 - DNA, 21 bp, Artificial sequence
  Non-active Sense sequence
guaguuggagcuguuggcgtt

SEQ ID NO: 156 - DNA, 21 bp, Artificial sequence
  Non-active Sense sequence
uaguuggagcuguuggcgutt

SEQ ID NO: 157 - DNA, 21 bp, Artificial sequence
  Non-active Sense sequence
aguuggagcuguuggcguatt

SEQ ID NO: 158 - DNA, 21 bp, Artificial sequence
  Non-active Sense sequence
guuggagcuguuggcguagtt

SEQ ID NO: 159 - DNA, 21 bp, Artificial sequence
  Non-active Sense sequence
uuggagcuguuggcguaggtt

SEQ ID NO: 160 - DNA, 21 bp, Artificial sequence
  Non-active Sense sequence
uggagcuguuggcguaggctt

SEQ ID NO: 161 - DNA, 21 bp, Artificial sequence
  Non-active Sense sequence
ggagcuguuggcguaggcatt

SEQ ID NO: 162 - DNA, 21 bp, Artificial sequence
  Non-active Sense sequence
gagcuguuggcguaggcaatt

SEQ ID NO: 163 - DNA, 21 bp, Artificial sequence
  Non-active Sense sequence
agcuguuggcguaggcaagtt

SEQ ID NO: 164 - DNA, 21 bp, Artificial sequence
  Non-active Sense sequence
gcuguuggcguaggcaagatt

SEQ ID NO: 165 - DNA, 21 bp, Artificial sequence
  Non-active Sense sequence
cuguuggcguaggcaagagtt

SEQ ID NO: 166 - DNA, 21 bp, Artificial sequence
  Non-active Sense sequence
uguuggcguaggcaagagutt

SEQ ID NO: 167 - DNA, 21 bp, Artificial sequence
  Non-active Sense sequence
guuggcguaggcaagagugtt

SEQ ID NO: 168 - DNA, 21 bp, Artificial sequence
  Non-active Sense sequence
uuggcguaggcaagagugctt

SEQ ID NO: 169 - DNA, 21 bp, Artificial sequence
  Non-active Sense sequence
uggcguaggcaagagugcctt

SEQ ID NO: 170 - DNA, 21 bp, Artificial Sequence
  Non-active Sense sequence
ggcguaggcaagagugccatt

SEQ ID NO: 171 - DNA, 51 bp, Artificial Sequence
  screening oligonucleotide
aattccatatgcttgtggtagttggagctgttggcgtaggcaagagtgcca

SEQ ID NO: 172 - DNA, 51 bp, Artificial Sequence
  screening oligonucleotide
gatctggcactcttgcctacgccaacagctccaactaccacaagcatatgg

SEQ ID NO: 173 - DNA, 51 bp, Artificial Sequence
  screening oligonucleotide
aattccatatgcttgtggtagttggagctggtggcgtaggcaagagtgcca

SEQ ID NO: 174 - DNA, 51 bp, Artificial Sequence
  screening oligonucleotide
gatctggcactcttgcctacgccaccagctccaactaccacaagcatatgg

SEQ ID NO: 175 - DNA, 21 bp, Artificial sequence
  siRNA sequence for potential off target
gactacaaggacgacgatgac

SEQ ID NO: 176 - DNA, 21 bp, Artificial sequence
  siRNA sequence for potential off target
cccctacgacgtgcccgacta

SEQ ID NO: 177 - DNA, 21 bp, Artificial sequence
  siRNA sequence for potential off target
ccacagatgtgaagttcattt

SEQ ID NO: 178 - DNA, 21 bp, Artificial sequence
  siRNA sequence for potential off target
aacaccagctcctgtgctgcg

SEQ ID NO: 179 - DNA, 21 bp, Artificial Sequence
  siRNA sequence for potential off target
caggaacagattgaagccctt

SEQ ID NO: 180 - DNA, 41 bp, Artificial Sequence
  G12V-Endotag
acttgtggtagttggagctgttggcgtaggcaagagtgcct

SEQ ID NO: 181 - DNA, 41 bp, Artificial Sequence
  WT-Endotag
acttgtggtagttggagctggtggcgtaggcaagagtgcct

SEQ ID NO: 182 - DNA, 42 bp, Artificial Sequence
  Flag-Ha-CycD1-Stop-V5-Tag
ggcaaaccgattccgaacccgctgctgggcctggatagcacc

SEQ ID NO: 183 - RNA, 21 nt, Artificial sequence
  Active antisens
ucuguagcuagaccaaaauca

SEQ ID NO: 184 - RNA, 21 nt, Artificial sequence
  Active antisens
cucuguagcuagaccaaaauc

SEQ ID NO: 185 - RNA, 21 nt, Artificial sequence
  Active antisens
ucucuguagcuagaccaaaau

SEQ ID NO: 186 - RNA, 21 nt, Artificial sequence
  Active antisens
uucucuguagcuagaccaaaa

SEQ ID NO: 187 - RNA, 21 nt, Artificial sequence
  Active antisens
uuucucuguagcuagaccaaa

SEQ ID NO: 188 - RNA, 21 nt, Artificial sequence
  Active antisens
auuucucuguagcuagaccaa

SEQ ID NO: 189 - RNA, 21 nt, Artificial sequence
  Active antisens
gauuucucuguagcuagacca

SEQ ID NO: 190 - RNA, 21 nt, Artificial sequence
  Active antisens
agauuucucuguagcuagacc

SEQ ID NO: 191 - RNA, 21 nt, Artificial sequence
  Active antisens
gagauuucucuguagcuagac

SEQ ID NO: 192 - RNA, 21 nt, Artificial sequence
  Active antisens
cgagauuucucuguagcuaga

SEQ ID NO: 193 - RNA, 21 nt, Artificial sequence
  Active antisens
ucgagauuucucuguagcuag

SEQ ID NO: 194 - RNA, 21 nt, Artificial sequence
  Active antisens
aucgagauuucucuguagcua

SEQ ID NO: 195 - RNA, 21 nt, Artificial sequence
  Active antisens
caucgagauuucucuguagcu

SEQ ID NO: 196 - RNA, 21 nt, Artificial sequence
  Active antisens
ccaucgagauuucucuguagc

SEQ ID NO: 197 - RNA, 21 nt, Artificial sequence
  Active antisens
uccaucgagauuucucuguag

SEQ ID NO: 198 - RNA, 21 nt, Artificial sequence
  Active antisens
cuccaucgagauuucucugua

SEQ ID NO: 199 - RNA, 21 nt, Artificial sequence
  Active antisens
acuccaucgagauuucucugu

SEQ ID NO: 200 - RNA, 21 nt, Artificial sequence
  Active antisens
cacuccaucgagauuucucug

SEQ ID NO: 201 - RNA, 21 nt, Artificial sequence
  Active antisens
ccacuccaucgagauuucucu

SEQ ID NO: 202 - RNA, 21 nt, Artificial sequence
  Active antisens
cccacuccaucgagauuucuc

SEQ ID NO: 203 - RNA, 21 nt, Artificial sequence
  Active antisens
acccacuccaucgagauuucu

SEQ ID NO: 204 - DNA, 21 bp, Artificial sequence
  non active sens sequence
auuuuggucuagcuacagatt

SEQ ID NO: 205 - DNA, 21 bp, Artificial sequence
  non active sens sequence
uuuuggucuagcuacagagtt

SEQ ID NO: 206 - DNA, 21 bp, Artificial sequence
  non active sens sequence
uuuggucuagcuacagagatt

SEQ ID NO: 207 - DNA, 21 bp, Artificial sequence
  non active sens sequence
uuggucuagcuacagagaatt

SEQ ID NO: 208 - DNA, 21 bp, Artificial sequence
  non active sens sequence
uggucuagcuacagagaaatt

SEQ ID NO: 209 - DNA, 21 bp, Artificial sequence
  non active sens sequence
ggucuagcuacagagaaautt

SEQ ID NO: 210 - DNA, 21 bp, Artificial sequence
  non active sens sequence
gucuagcuacagagaaauctt

SEQ ID NO: 211 - DNA, 21 bp, Artificial sequence
  non active sens sequence
ucuagcuacagagaaaucutt

SEQ ID NO: 212 - DNA, 21 bp, Artificial sequence
  non active sens sequence
cuagcuacagagaaaucuctt

SEQ ID NO: 213 - DNA, 21 bp, Artificial sequence
  non active sens sequence
uagcuacagagaaaucucgtt

SEQ ID NO: 214 - DNA, 21 bp, Artificial sequence
  non active sens sequence
agcuacagagaaaucucgatt

SEQ ID NO: 215 - DNA, 21 bp, Artificial sequence
  non active sens sequence
gcuacagagaaaucucgautt

SEQ ID NO: 216 - DNA, 21 bp, Artificial sequence
  non active sens sequence
cuacagagaaaucucgaugtt

SEQ ID NO: 217 - DNA, 21 bp, Artificial sequence
  non active sens sequence
uacagagaaaucucgauggtt

SEQ ID NO: 218 - DNA, 21 bp, Artificial sequence
  non active sens sequence
acagagaaaucucgauggatt

SEQ ID NO: 219 - DNA, 21 bp, Artificial sequence
  non active sens sequence
cagagaaaucucgauggagtt

SEQ ID NO: 220 - DNA, 21 bp, Artificial sequence
  non active sens sequence
agagaaaucucgauggagutt

SEQ ID NO: 221 - DNA, 21 bp, Artificial sequence
  non active sens sequence
gagaaaucucgauggagugtt

SEQ ID NO: 222 - DNA, 21 bp, Artificial sequence
  non active sens sequence
agaaaucucgauggaguggtt

SEQ ID NO: 223 - DNA, 21 bp, Artificial sequence
  non active sens sequence
gaaaucucgauggagugggtt

SEQ ID NO: 224 - DNA, 21 bp, Artificial sequence
  non active sens sequence
aaaucucgauggagugggutt

SEQ ID NO: 225 - DNA, 89 bp, Artificial sequence
  screening oligonucleotide
gatcccaattgtagttagtttagaccggttgattttggtctagctacagtgaaatctcgatggagtgggtacgcgtagatcttatttgc

SEQ ID NO: 226 - DNA, 89 bp, Artificial sequence
  screening oligonucleotide
ggccgcaaataagatctacgcgtacccactccatcgagatttcactgtagctagaccaaaatcaaccggtctaaactaactacaattgg

SEQ ID NO: 227 - DNA, 89 bp, Artificial sequence
  screening oligonucleotide
gatcccaattgtagttagtttagaccggttgattttggtctagctacagagaaatctcgatggagtgggtacgcgtagatcttatttgc

SEQ ID NO: 228 - DNA, 89 bp, Artificial sequence
  screening oligonucleotide
ggccgcaaataagatctacgcgtacccactccatcgagatttctctgtagctagaccaaaatcaaccggtctaaactaactacaattgg

SEQ ID NO: 229 - DNA, 41 bp, Artificial sequence
  V600E Raf endotag
tgattttggtctagctacagagaaatctcgatggagtgggt

SEQ ID NO: 230 - DNA, 41 bp, Artificial sequence
  WT Raf endotag
tgattttggtctagctacagtgaaatctcgatggagtgggt

(19) United States
(12) Patent Application Publication
(10) Pub. No.: US 2019/0345212 A1
(43) Pub. Date: Nov. 14, 2019

(54) HYBRID NUCLEIC ACID MOLECULES AND THEIR USE

(71) Applicants: CENTRE NATIONAL DE LA RECHERCHE SCIENTIFIQUE, Paris (FR); UNIVERSITE DE MONTPELLIER, Montpellier (FR); INSTITUT NATIONAL DE LA SANTE ET DE LA RECHERCHE MEDICALE(INSERM), Paris Cedex 13 (FR)

(72) Inventor: Frederic BIENVENU, Saint-Clement-De-Riviere (FR)

(21) Appl. No.: 16/478,633

(22) PCT Filed: Jan. 18, 2018
(86) PCT No.: PCT/EP2018/051214

§ 371 Date: Jul. 17, 2019

(30) Foreign Application Priority Data
Jan. 18, 2017 (EP) 17305056.8

(51) Int. Cl. C07K 14/47; C12N 15/11; C12N 15/62; A01K 67/027; G01N 33/50; A61K 49/00

(52) U.S. Cl. CPC C07K 14/4738; C12N 15/111; C12N 15/62; A01K 67/0275; G01N 33/5011; A61K 49/0008; A01K 2267/0331; C12N 2320/11; C12N 2330/31; C12N 2740/10043; C07K 2319/00; A01K 2207/12; A01K 2227/105; C12N 2310/14

(57) ABSTRACT

The invention relates to a nucleic acid molecule comprising: a. a first region comprising a nucleic acid sequence coding M for the protein Cyclin D1, also called CCND1, said first region being controlled by means allowing the expression of said protein, and b. at least one second region, said second region comprising essentially a sequence from 14 to 59 nucleic acids, said second region corresponding to a transcribed region of a gene, said second region containing at least a genetic modification compared to the same region of the corresponding wild-type version of said gene, said second region being genetically isolated from the means allowing the expression of said protein such that said second region is not translated into a peptide.

The invention relates to hybrid nucleic acid molecules and their use.

RNA interference (RNAi) consists in the targeting of messenger RNA by endogenous (micro RNA) or ectopic small interfering RNA (siRNA). This leads to mRNA degradation and/or the inhibition of mRNA translation.

RNAi is considered as a modem therapeutic solution against numerous disorders with several promising clinical trials already initiated.

One major caveat of RNAi is the “off-target” effect, which can contribute to the functional differences observed in comparison with genetic ablation experiments.

Furthermore, uncontrolled artificial “off-target” interference of genetic nodes can subversively re-create a functional phenotype which should not be attributed solely to the primary target. Of particular concern in gene expression profiling experiments, the “off-targeting” of transcription regulators could induce a myriad of transcriptional bias genome-wide. To exclude such “off-target” noise, it is mandatory to perform rescue experiments with versions of the targeted gene that are “resistant” to the siRNA tested as an appropriate control.

In practice however, many rescue experiments deviate cell integrity due to the non physiological over-expression of the transgene of interest or because of its toxicity. To simplify, such fastidious controls are rarely used in vivo.

So there is a need to overcome these inconvenient.

One aim of the invention is to provide a molecule that could help to screen siRNA that exclude off-targets noise.

Another aim of the invention is to provide a method for rapidly and efficiently select such siRNA, and which is cost effective.

The invention relates to a nucleic acid molecule comprising:
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said second region containing at least one genetic modification or alteration compared to the same region of the corresponding wild-type version of said gene, said second region being genetically isolated from the means allowing the expression of said protein such that said second region is not translated into a peptide.

In other words, the invention relates to a nucleic acid molecule comprising:
a first region comprising a nucleic acid sequence coding for the protein Cyclin D1, also called CCND1, said first region being controlled by means allowing the expression of said protein, and
   at least one second region, said second region comprising essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide.

The invention is based on the unexpected observation made by the inventors that the use of cyclin D1 may be a good marker for identifying compounds liable to promote RNA interference. Moreover, the hybrid nucleic acid construction (i.e. hybrid nucleic acid molecule) according to the invention is also very powerful to identify such a compound and to identify and possibly overcome off-targets due to RNA interference.

In the invention, the nucleic acid molecule comprise or consists essentially of two specific regions: a first region which codes for a reporter, which is the cyclin D1 protein, and a second region which represents the target for a compound, formally a small inhibiting RNA (siRNA). The properties of the nucleic acid according to the invention are that without siRNA, cyclin D1 is expressed and exert a proliferative and an anti-apoptotic effect on cells into which it is expressed. When a siRNA is specifically targeted to the second region, then the RNA interference can be carried out, and the result is that the mRNA corresponding to the nucleic acid molecule according to the invention is therefore destroyed or its translation is inhibited. As a consequence, the cyclin D1 protein is not expressed and therefore not able to protect anymore the cells from apoptosis.

Thus, when a siRNA is specific to a target contained in the nucleic acid molecule according to the invention, i.e. specific to the second region, cell death can occur, and it is possible to state that the siRNA is functional and recognizes at least the target.

Other readouts can be studied such as proliferation, cell survival, cell migration, metabolic function or simply phosphorylation of targets of the CDK4/6-cyclin D1 complex. Thus, any modification of the homeostasis of a cell expressing the nucleic acid molecule according to the invention, and treated with a specific siRNA is a hallmark that said siRNA is functional regarding this target, i.e. has hybridized to the second region.

One particular advantage of the nucleic acid molecule according to the invention is to allow the screening of siRNA which are specific to mutated sequences occurring in several pathologies. Indeed, one of the aims of the invention is to propose a new way to screen siRNA that could be used to specifically inactivate (or to specifically reduce the expression) of abnormal RNAs or proteins resulting from a genetic alteration, and that exert an abnormal function in the cell. Thus, a therapy using said screened siRNA could be envisaged since the selected siRNA would not affect the natural counterpart of said gene, and only the cause of the pathology would or will be eliminated.

In the invention, “genetic alteration” means any nucleic acid modification, within a nucleic acid molecule, by at least one substitution, an insertion or a deletion of at least one nucleotide, compared to the wild type sequence. This also encompasses any chromosomal translocation or gene fusion which results to the formation of a hybrid nucleic molecule not naturally occurring in healthy or wildtype animals, including human.

In the invention, the nucleic acid molecule is either a DNA molecule or a RNA molecule.

Thus, the first sequence or region of the nucleic acid according to the invention contains the sequence coding for the human or murine cyclin D1 protein. This protein is very useful for its anti-apoptotic properties, but also because of its very short half-life which is about 20 minutes. Therefore, when RNA interference occurs, cyclin D1 has completely disappeared after only few hours, and the biological effects of its absence are easily detectable or measurable.

The first region contains therefore the complete open reading frame of murine or human cyclin D1 gene, and means allowing the expression of said proteins, i.e. at least sequences allowing the translation of said proteins. If the nucleic acid molecule according to the invention is a DNA molecule, it is relevant that the molecule also contain means allowing the transcription into RNA of said nucleic acid molecule.

The second region of the nucleic acid molecule of the invention contains the target for siRNA that have to be screened. This region comprises from 14 to 59 nucleic acids, which means that this region comprises 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 nucleotides.

The second region contains the sequence of a mutated gene, said sequence containing the mutation, preferably approximately in the middle of the sequence. For instance, if the second region contains 14 nucleotides, the mutation (for instance a substitution), would be positioned at position 7 or 8 of the sequence.

It is also important that the second region corresponds to a part of the mutated gene which contains 1) a mutation, but also 2) which is expressed in RNA, i.e. which is transcribed. Thus, a sequence of a mutated gene which would be located for instance in untranscribed transcription regulatory elements are not included in the second region according to the invention.

Another important point regarding the second region is that such region, even it is transcribed when the nucleic acid molecule is a DNA molecule, said second region must not be translated into a peptide. Indeed, the inventors noticed that when they are expressed some mutated portions of a gene, produce a reduced-size peptide which can per se induce phenotypical changes in cells, changes that are similar or closely identical to the effect of the full length mutated protein. Thus, it is important that the second region be genetically isolated from the translation machinery in order to avoid any translating of said second region.

It is important to notice, within the frame of the invention, that the genetic modification occurring in the transcribed region of a gene contained in the second region does not participate to the genetic isolation, i.e. is not responsible of the inhibition of the translation of the second region.

To summarize, the nucleic acid molecule according to the invention is either a RNA molecule or a DNA molecule which is integrally transcribed. In other words, the nucleic acid molecule according to the invention is either a RNA molecule or a DNA molecule which can be transcribed but never translated in its entirety. Only the region coding for the reporter, if transcribed, should be translated. However, regarding the translation, only the sequence of the cyclin D1 protein contained region 1 is expressed as a protein, whereas region 2 should not be translated into peptide.

Thus, the invention as defined above relates to a nucleic acid molecule comprising:
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide,

wherein said genetic isolation is carried out by a part which is different from said transcribed region of a gene containing at least a genetic modification and different from said at least a genetic modification.

In one particular embodiment, the invention relates to the above mentioned nucleic acid molecule comprising:
a first region comprising a nucleic acid sequence coding for the protein Cyclin D1, also called CCND1, said first region being controlled by means allowing the expression of said protein, and
at least one second region, said second region comprising or consisting essentially of a sequence from 14 to 59 nucleic acids, said second region corresponding to a transcribed region of the gene coding for said cyclin D1 protein, said second region containing at least a genetic modification compared to the same region of the corresponding wild-type version of said gene, said second region being genetically isolated from the means allowing the expression of said protein such that said second region is not translated into a peptide.

Advantageously, the invention relates to the nucleic acid molecule as defined above, in which said first region comprises one of the following sequences coding for said CCND1 protein as set forth in SEQ ID NO: 1 or SEQ ID NO: 2.

SEQ ID NO: 1 is a DNA sequence representing the open reading frame of the cyclin D1 protein originating from human. SEQ ID NO: 1 codes for the protein as set forth in SEQ ID NO: 3, from the RNA as set forth in SEQ ID NO: 26.

SEQ ID NO: 2 is a DNA sequence representing the open reading frame of the cyclin D1 protein originating from mouse. SEQ ID NO: 2 codes for the protein as set forth in SEQ ID NO: 4, from the RNA as set forth in SEQ ID NO: 27.

When the nucleic acid according to the invention is a RNA molecule, said first region comprises one of the following sequences as set forth in SEQ ID NO: 26 or SEQ ID NO: 27, coding for said CCND1 protein.

Advantageously, the invention relates to the above mentioned nucleic acid molecule, wherein said means allowing expression of said CCND1 protein are means allowing translation initiation by ribosomes.

As mentioned above, the sequence coding for cyclin D1 protein is translated into protein by means allowing the expression, i.e. the synthesis, of the protein. These means are sequences allowing at first 1) the loading of ribosomes onto the messenger RNA molecule that has been previously transcribed. Such sequences are Internal Ribosome Entry Sites (IRES) sequences, for instance as set forth in SEQ ID NO: 28 or 29, or five-prime cap (5′ cap). Second is 2) a sequence inducing the initiation of translation by ribosomes. Such sequences are for instance the so-called “KOZAK sequences” containing the sequence AccAUGG or AccATGG, and derivatives,

This means or sequences allowing the expression of said cyclin D1 protein are located in 5′ position such that 1) is followed by 2) compared to the sequence of said cyclin D1 protein, and exert a cis regulation effect.

More advantageously, the invention relates to the nucleic acid molecule above-defined, wherein said first region comprises or consists essentially of one of sequences as set forth in SEQ ID NO: 5, SEQ ID NO: 6, SEQ ID NO: 7, SEQ ID NO: 8, SEQ ID NO: 9 or SEQ ID NO: 10.

When the nucleic acid molecule according to the invention is a RNA molecule said first region comprises or consists essentially of one of sequences as set forth in SEQ ID NO: 30, SEQ ID NO: 31, SEQ ID NO: 32, SEQ ID NO: 33, SEQ ID NO: 34 or SEQ ID NO: 35.

Advantageously, the invention relates to the nucleic acid as defined above, wherein said first region is located in a 5′ position of said second region.

Advantageously, the invention relates to the nucleic acid as defined above, wherein said first region is located in a 3′ position of said second region.

According to the invention, the first and the second region are linked but the first region can be uniformly positioned in position 5′ or 3′ of the second region. It is only important that the means allowing the expression of the sequence of Cyclin D1 contained in the first region does not allow the translation of the second region. So, the genetic isolation is important, but not the position of the first region compared to the second region.

In one advantageous embodiment, the invention relates to the nucleic acid molecule as defined above, wherein said second region is genetically isolated from said first region by at least one sequence of end of translation.

To avoid a translation of the second region, it is relevant to place between the first region and the second region sequences of termination of the translation, possibly within the 3 reading frames. For instance, it could be relevant to add between the two regions a first stop codon, followed by one nucleotide and immediately downstream a second stop codon, followed by two nucleotides and immediately downstream a third stop codon. This succession of stop codon within the 3 reading frames will avoid any progression of the peptide synthetizing ribosomes, and will isolate genetically the second region from translation.

In one advantageous embodiment, the invention relates to the nucleic acid as defined above, said nucleic acid molecule comprising or consisting essentially of the sequences as set forth in SEQ ID NO: 11, SEQ ID NO: 15, and SEQ ID NO: 19, when the nucleic acid molecule is a DNA molecule.

In one advantageous embodiment, the invention relates to the nucleic acid as defined above, said nucleic acid molecule comprising or consisting essentially of the sequences as set forth in SEQ ID NO: 36, SEQ ID NO: 40 and SEQ ID NO: 44, when the nucleic acid molecule is a DNA molecule.

SEQ ID NO: 11, 15, 36 and 40 represent molecules according to the invention wherein the second region contains a target for siRNA that specifically recognize a mutation within the mutated sequence of the oncogenic form of Kras gene: KRASG12V.

SEQ ID NO: 19 and 44 represent molecules according to the invention wherein the second region contains a target for siRNA that specifically recognize a mutation within the mutated sequence of the oncogenic form of Braf gene: BRAFV600E.

The invention also relates to a cell, possibly a tumoral cell, comprising at least one copy of the nucleic acid molecule as defined above.

The cell according to the invention, which comprises the nucleic acid molecule as defined above, may be a “normal” cell, i.e. a cell having no pathologic features and with a limited life time. The cell according to the invention may also be a tumoral cell, i.e. a cell having one or more hallmarks of cancer.

The cell may also be a cell, tumoral or not, that does not express, due to mutation or deletion, the endogenous cyclin D1 protein expressing gene.

According to the invention, the abovementioned cells contain at least one nucleic acid molecule as defined above. This at least one nucleic acid molecule is either present in a form of free molecule (possibly inserted in a vector as an episome), or inserted into the cellular DNA (i.e. inserted into the genome of said cells).

The invention also relates to a genetically modified non-human animal, preferably a rodent, in particular a rat or a mouse, comprising at least one cell as defined above.

The genetically modified non-human animal according to the invention may comprise at least one cell as defined above, for instance further to an injection or a graft. This animal may also contain one or more complete organs constituted by cells as defined above.

The invention also encompasses animals having all their cells as defined above. This is possible by common technics of transgenesis and cloning technics, possibly which are not an essentially biological process, e.g. the mating of male and female animals.

The invention also relates to a transgenic non-human animal having a modified endogenous CCND1 coding gene,

said gene being modified
either by the insertion, directly upstream of the translation initiation sequence containing the first ATG of the first exon of said CCND1 gene, a sequence consisting of at least one second region,
or by the insertion, directly downstream of the translation termination sequence containing the stop codon of the last exon of said CCND1 gene, a sequence consisting of at least one second region,

wherein said second region comprises essentially a sequence from 14 to 59 nucleic acids, said second region corresponding to a transcribed region of a gene, said second region containing at least a genetic modification compared to the same region of the corresponding wild-type version of said gene, said second region being genetically isolated from the means allowing the expression of said protein such that said second region is not translated into a peptide.

The invention also relates to a transgenic non-human animal having a modified endogenous CCND1 coding gene,

said gene being modified
either by the insertion, directly upstream of the translation initiation sequence containing the first ATG of the first exon of said CCND1 gene, a sequence consisting of at least one second region,
or by the insertion, directly downstream of the translation termination sequence containing the stop codon of the last exon of said CCND1 gene, a sequence consisting of at least one second region,

wherein said second region comprises essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide.

According to the invention, it is also possible to obtain a genetically modified animal that contains, at the locus of the endogenous gene coding for the Cyclin D1 protein, a modification in order to insert the region 2 as defined above.

The region is either
inserted in the 5′ end of the gene, within the transcribed region of the gene, but said second region being genetically isolated from the means allowing the translation of the endogenous cyclin D1 protein
inserted in the 3′ end of the gene, within the transcribed region of the gene, but said second region being genetically isolated from the means allowing the translation of the endogenous cyclin D1 protein.

The invention also relates to a subset of nucleic acid molecules, comprising
a first nucleic acid molecule as defined above, and
a second nucleic acid molecule, said second nucleic acid molecule comprising,
   i. The same first region compared to the first region of said first nucleic acid molecule, and possibly
   ii. At least a second region, said second region comprising essentially a sequence from 14 to 59 nucleic acids, said second region corresponding to a transcribed region of a gene, said second region comprising the wild-type version of said gene compared to the second region of said first nucleic acid molecule, said second region being genetically isolated from the means allowing the expression of said protein such that said second region is not translated into a peptide.

The inventors have also made the unexpected observation that a combination of a nucleic acid molecule according to the invention, and as defined above, and a second nucleic acid molecule having the same first region, i.e. a first region coding for a Cyclin D1 protein, but differing only by the mutation of the second region (i.e. corresponding to the wild type counterpart of the sequence contained in the second region of the first molecule), they can identify more precisely the siRNA that are specific of the second region of the first nucleic acid molecule.

The second region may also be absent in said second nucleic acid molecule.

In other words, the invention relates to a composition, i.e. a subset, comprising: either

a first nucleic acid molecule comprising
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said second region containing at least a genetic modification compared to the same region of the corresponding wild-type version of said gene, said second region being genetically isolated from the means allowing the expression of said protein such that said second region is not translated into a peptide, and

a second nucleic acid molecule comprising
the first region comprising a nucleic acid sequence coding for the protein Cyclin D1, also called CCND1, as seen in the first nucleic acid molecule, said first region being controlled by means allowing the expression of said protein,

or

a first nucleic acid molecule comprising
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said second region containing at least a genetic modification compared to the same region of the corresponding wild-type version of said gene, said second region being genetically isolated from the means allowing the expression of said protein such that said second region is not translated into a peptide, and

a second nucleic acid molecule comprising
the first region comprising a nucleic acid sequence coding for the protein Cyclin D1, also called CCND1, as seen in the first nucleic acid molecule, said first region being controlled by means allowing the expression of said protein, and
at least one second region, said second region comprising essentially a sequence from 14 to 59 nucleic acids, said second region corresponding to a transcribed region of a gene, said second region containing the part of the wild-type version of said gene, i.e. the same part without the genetic modification contained in the second region of said first nucleic acid molecule, said second region being genetically isolated from the means allowing the expression of said protein such that said second region is not translated into a peptide.

Advantageously, the invention relates to the subset as defined above, in which said first regions comprise one of the following sequences coding for said CCND1 protein as set forth in SEQ ID NO: 1 or SEQ ID NO: 2.

SEQ ID NO: 1 is a DNA sequence representing the open reading frame of the cyclin D1 protein originating from human. SEQ ID NO: 1 codes for the protein as set forth in SEQ ID NO: 3, from the RNA as set forth in SEQ ID NO: 26.

SEQ ID NO: 2 is a DNA sequence representing the open reading frame of the cyclin D1 protein originating from mouse. SEQ ID NO: 2 codes for the protein as set forth in SEQ ID NO: 4, from the RNA as set forth in SEQ ID NO: 27.

When the nucleic acid according to the invention is a RNA molecule, said first region comprises one of the following sequences as set forth in SEQ ID NO: 26 or SEQ ID NO: 27, coding for said CCND1 protein.

More advantageously, the invention relates to the subset above-defined, wherein said first regions comprise or consist essentially of one of sequences as set forth in SEQ ID NO: 5, SEQ ID NO: 6, SEQ ID NO: 7, SEQ ID NO: 8, SEQ ID NO: 9 or SEQ ID NO: 10.

When the nucleic acid molecules according to the invention is a RNA molecule said first region comprises or consists essentially of one of sequences as set forth in SEQ ID NO: 30, SEQ ID NO: 31, SEQ ID NO: 32, SEQ ID NO: 33, SEQ ID NO: 34 or SEQ ID NO:35.

In one advantageous embodiment, the invention relates to the above defined subset wherein:
said first nucleic acid molecule comprises or consists essentially of one of the sequences as set forth in SEQ ID NO: 11, 15, 19,
said second nucleic acid molecule comprises or consists essentially of one of the sequences as set forth in SEQ ID NO: 12 and 18,

More precisely, the invention relates to the following specific subsets:
a set comprising the nucleic acid molecules comprising or consisting essentially of the sequences as set forth in SEQ ID NO: 11 and 12, or
a set comprising the nucleic acid molecules comprising or consisting essentially of the sequences as set forth in SEQ ID No: 18 and 19.

When the nucleic acid molecules are RNA molecules, the invention relates to the above defined subset wherein:
said first nucleic acid molecule comprises or consists essentially of one of the sequences as set forth in SEQ ID NO: 36, 40, 44, 49,
said second nucleic acid molecule comprises or consists essentially of one of the sequences as set forth in SEQ ID NO: 37, 41, 43, 48,

More precisely, the invention relates to the following specific subsets:
a set comprising the nucleic acid molecules comprising or consisting essentially of the sequences as set forth in SEQ ID NO: 36 and 37, or
a set comprising the nucleic acid molecules comprising or consisting essentially of the sequences as set forth in SEQ ID No: 48 and 49.

In one other aspect, the invention also relates to a set of nucleic acid molecules, comprising:

i. a subset according as defined above,

ii. a third nucleic acid molecule, said third nucleic acid molecule comprising,
a first region comprising a nucleic acid sequence coding for a reporter protein other than CCND1, i.e. different from CCND1, said first region being controlled by means allowing translation of said reporter protein, and
A second region corresponding to the second region found in the first nucleic acid molecule, and

iii. a fourth nucleic acid molecule comprising,
A first region corresponding to the first region of said third nucleic acid molecule, and possibly
At least a second region, said second region comprising essentially a sequence from 14 to 59 nucleic acids, said second region corresponding to a transcribed region of a gene, said second region comprising the wild-type version of said gene compared to the second region of said first or third nucleic acid molecule, said second region being genetically isolated from the means allowing the expression of said protein such that said second region is not translated into a peptide.

The inventors also found that, if two sequences are used, in addition to the subset defined above, it is possible to determine if the second region i.e. the said second region corresponding to a genetic alteration, may modify the homeostasis of the cell without necessarily being translated into a peptide. So they decided to add two additional nucleic acid molecules, i.e. a third and a fourth nucleic acid molecule, corresponding to the first and the second nucleic acid molecule wherein the first region codes for a protein which is not the cyclin D1 protein, but another reporter.

The reporter that can be contained in the first regions of both the third and the fourth nucleic acid molecule of the above defined set can be selected from the well-known in the art reporters, such as short protein tags, fluorescent and bioluminescent proteins, immunoglobulin . . . . This list is not limitative, and the skilled person could easily choose the best reporter for this purpose.

In other words, the invention relates to a set comprising

a first nucleic acid molecule comprising
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide, and

a second nucleic acid molecule comprising
the first region comprising a nucleic acid sequence coding for the protein Cyclin D1, also called CCND1, as seen in the first nucleic acid molecule, said first region being controlled by means allowing the expression of said protein, and possibly
at least one second region, said second region comprising essentially a sequence from 14 to 59 nucleic acids, said second region corresponding to a transcribed region of a gene, said second region containing a part of the wild-type version of said gene, i.e. without the genetic modification contained in the second region of said first nucleic acid molecule, said second region being genetically isolated from the means allowing the expression of said protein such that said second region is not translated into a peptide,

a third nucleic acid molecule comprising
a first region comprising a nucleic acid sequence coding for a reporter protein, which is not the cyclin D1 protein, said first region being controlled by means allowing the expression of said protein, and
at least one second region, said second region comprising essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide, and

a fourth nucleic acid molecule comprising
a first region comprising a nucleic acid sequence coding for a reporter protein, which is not the cyclin D1 protein, said first region of said fourth nucleic acid molecule being identical to the first region of said third region, said first region being controlled by means allowing the expression of said protein, and possibly
at least one second region, said second region comprising essentially a sequence from 14 to 59 nucleic acids, said second region corresponding to a transcribed region of a gene, said second region containing a part of the wild-type version of said gene, i.e. without the genetic modification contained in the second region of said first or third nucleic acid molecule, said second region being genetically isolated from the means allowing the expression of said protein such that said second region is not translated into a peptide.

Advantageously, the invention relates to the set as defined above, in which said first regions of said first and second nucleic acid molecules comprise one of the following sequences coding for said CCND1 protein as set forth in SEQ ID NO: 1 or SEQ ID NO: 2.

SEQ ID NO: 1 is a DNA sequence representing the open reading frame of the cyclin D1 protein originating from human. SEQ ID NO: 1 codes for the protein as set forth in SEQ ID NO: 3, from the RNA as set forth in SEQ ID NO: 26.

SEQ ID NO: 2 is a DNA sequence representing the open reading frame of the cyclin D1 protein originating from mouse. SEQ ID NO: 2 codes for the protein as set forth in SEQ ID NO: 4, from the RNA as set forth in SEQ ID NO: 27.

When the nucleic acid according to the invention is a RNA molecule, said first region comprises one of the following sequences as set forth in SEQ ID NO: 26 or SEQ ID NO: 27, coding for said CCND1 protein.

More advantageously, the invention relates to the subset above-defined, wherein said first regions comprise or consist essentially of one of sequences as set forth in SEQ ID NO: 5, SEQ ID NO: 6, SEQ ID NO: 7, SEQ ID NO: 8, SEQ ID NO: 9 or SEQ ID NO: 10.

When the nucleic acid molecules according to the invention is a RNA molecule said first region comprises or consists essentially of one of sequences as set forth in SEQ ID NO: 30, SEQ ID NO: 31, SEQ ID NO: 32, SEQ ID NO: 33, SEQ ID NO: 34 or SEQ ID NO:35.

In one advantageous embodiment, the invention relates to the above defined subset wherein:
said first nucleic acid molecule comprises or consists essentially of one of the sequences as set forth in SEQ ID NO: 11, 15, or 19,
said second nucleic acid molecule comprises or consists essentially of one of the sequences as set forth in SEQ ID NO: 12, or 18,
said third nucleic acid molecule comprises or consists essentially of one of the sequences as set forth in SEQ ID NO: 17 or 24 and
said fourth nucleic acid molecule comprises or consists essentially of one of the sequences as set forth in SEQ ID NO: 16 or 23.

More precisely, the invention relates to the following specific subsets:
a set comprising the nucleic acid molecules comprising or consisting essentially of the sequences as set forth in SEQ ID NO: 11, 12, 23 and 24 or
a set comprising the nucleic acid molecules comprising or consisting essentially of the sequences as set forth in SEQ ID NO: 11, 12, 24 and 25 or
a set comprising the nucleic acid molecules comprising or consisting essentially of the sequences as set forth in SEQ ID NO: 19, 18, 17 and 16 or
a set comprising the nucleic acid molecules comprising or consisting essentially of the sequences as set forth in SEQ ID NO: 19, 18, 17 and 25.

The invention also relates to the use of
at least a nucleic acid molecule as defined above, or
a subset as defined above, or
a set as defined above,

for the in vitro screening of small interfering nucleic acid molecules.

As mentioned above, the nucleic acid molecule, the subset or the set as defined above are useful to screen siRNA specific of the second region of said nucleic acid molecules, and allows to screen specific siRNA limiting or avoiding off-targets in a cost effective manner.

The invention also relates to a method for screening, possibly in vitro, small interfering nucleic acid molecules comprising a step of contacting a tumoral cell containing
a nucleic acid molecule as defined above, or
a subset as defined above, or
a set as defined above,

with small interfering nucleic acid molecules, and

a step of evaluating said tumoral cell homeostasis.

According to the above method, and in order to determine if a siRNA to be screened is acceptable according to the criteria defined in the invention, it is possible to evaluate the homeostasis of the cell (as defined above).

Advantageously, the invention relates to the above method, for in vitro identifying the tumoral effect of nucleic acid sequence containing a genetic modification compared to its wild type counterpart, said method comprising a step of contacting a tumoral cell containing a set as defined above with small interfering nucleic acid molecules.

The invention also relates to a method for screening small interfering nucleic acid molecules comprising:
a step of injecting tumoral cells comprising a nucleic acid molecule as defined above into an immunosuppressed non-human animal, possible an immunosuppressed mouse or rat, in order to allow a tumor growth,
a step of injecting into the growing tumor a small interfering nucleic acid molecule at least complementary to the second region contained in said nucleic acid molecule, and
a step of selecting the small interfering nucleic acid molecule allowing a tumor regression.

As shown in the example, it is possible to subcutaneously graft some cells in the flanck of an immunosuppressed mouse, such that the cell will grow and develop a tumor at the injection point (in particular due to the expression of the cyclin D1 protein).

When treated with siRNA to be screened, if the siRNA is specific to said region 2, the cyclin D1 will disappear, and the tumor will eventually rapidly regress, or show hallmarks of biological homeostasis changes.

As a consequence, if a siRNA is able to inhibit the tumor growth, or to induce a tumoral regression, it would be considered as a good siRNA candidate.

The invention will better understand in view of the following example and the figures detailed hereafter.

### BRIEF DESCRIPTION OF THE DRAWINGS

FIGS. 1-6—Functional hyper-specificity of TAG-RNA

FIG. 1 represents the TAG-RNAi design to target tagged-Cyclin D1 mRNA (large arrow) but spare wildtype Cyclin D1. Flag tag is represented in black; HA tag in grey and Ccnd1 coding exons (numbered) or Untranslated Region (UTR) in white. WT mRNA is unaffected by TAG-RNAi but shares the similar off-target functional impact than Tagged responding cells.

FIG. 2 represents an immunoblot using anti-cyclin D1 (i.) or anti-actin (ii.) antibodies of RAS-G12V/DNP53 transformed MEFs of Ccnd1^{+/+} (1.), Ccnd1Ntag/Ntag (2.) or Ccnd1 Ctag/Ctag (3.) genotype treated with scramble (A) Flag (B) or HA (C) TAG-siRNA.

FIG. 3 represents the Venn diagrams representing the genes differentially expressed and their degree of overlap within each other (expressed as % of similarity) after RNA interference using siRNAs specific to CycD1 in RAS-G12V/DNP53 transformed MEFs of Ccnd1Ctag/Ctag genotype. Nat corresponds to a previously described custom made siRNA24, Qia corresponds to a commercial siRNA sequence provided by the Qiagen company and Life corresponds to a commercial siRNA sequence provided by the Life Technologies company (see Table 1). Arrows highlight the three other G1-Cyclins (putative off-targets) that are affected by some of these siRNAs but not by TAG-siRNAs from FIG. 4.

FIG. 4 represents Venn diagrams representing the genes differentially expressed after RNA interference using siRNAs specific to Flag or HA Tags in RAS-G12V/DNP53 transformed MEFs of Ccnd1Ctag/Ctag genotype. The arrow highlights Cyc-D2 which is the only other G1-Cyclin affected by the targeting of CycD1 using HA-RNAi.

FIG. 5 represents an immunoblot for G1-Cyclins by using anti-cyclin D1 (1), anti-cyclin D2 (2), anti-cyclin D3 (3b), anti-cyclin E1 (4) and anti CDK4 (5) antibodies (and controlled with anti-actin antibody (6) after RNA interference using three CycD1 “specific” siRNAs (Nat: D, Qui: E and Life: F), or Flag (B) and HA (C) siRNAs, in RAS-G12V/DNP53 transformed MEFs of Ccnd1 Ctag/Ctag genotype. Scramble siRNA treatment are shown in A

FIG. 6 represents a graph showing the in vivo RNAi functional impact on tumor burden dynamics of RAS-G12V/DNP53 transformed MEFs of Ccnd1^{-/-} genotype rescued by Tagged-CycD1 (curve with squares) or Untagged-CycD1 (curve with diamonds) transgene. Note that TAG-RNAi has no significant impact in absence of Tagged-CycD1 transgene (black curve). HA-siRNA was used for TAG-RNAi in this experiment. TAG-RNAi treatment (illustrated by the bar) was initiated on the morning of Day 0 (see methods). Values are represented as average tumor size in mm³ of n=5 tumors+/− standard error of the mean. X-axis: days, y-axis: average tumor burden in mm³.

**p<0.01; pairwise comparison using two-tailed paired Student's t test.

FIGS. 7-11—Endogenous mutation-specific TAG-RNAi

FIG. 7 represents an immunoblot from lysates of cells expressing versions of CycD1 transgene depicted on the right schematic. The black box is the nucleotide sequence encoding for FLAG tag, the grey box is the nucleotide sequence encoding for HA tag and the box is the Kozak sequence (K). i: Flag-CycD1-HA; ii: HA-CycD1-Flag; iii: Flag-K-CycD1-HA and iv: K-CycD1-HA-Stop-Flag. Cells are treated with scramble siRNA (A) or with Flag (B) or Ha (C) siRNAs. Proteins were labelled with anti-cyclin D1 (1) or actin (2) antibodies.

Note that the TAG-RNAi approach works equally when targeting the 5′ or the 3′ end of the mature messenger RNA and whether or not the genetic targeted TAG is translated as part of the coding sequence.

FIG. 8 is a schematic representing the generation of a TAG-RNAi strategy specific to the Kras mutation of the codon 12 (G12V-Endotag). The mutant G12V-Endotag (right dark grey) or the non-mutated WT-Endotag (right black) sequence spans from the −20 to the +20 nucleotides around the mutation and are fused to the non-coding part of the reporter gene encoding for Ntag (left black/grey)-CycD1.

FIG. 9 represents an histogram showing KRAS-G12V-Endotag specific knock down of the Ntag-CycD1 reporter constructs from FIG. 8 and measured by Tandem-HTRF (see methods), highlighting the major impact of the Ras-Endotag-siRNA#4 (C) on the expression of the mRNA carrying the mutation (grey bars) while only minor effect is observed on the mRNA carrying the non-mutated nucleotide sequence (black bars). Scramble-siRNA (Scr) is used as a negative control (A), HA-siRNA (B) is used as a positive control and Ras-Endotag-siRNA#12 (D) which targets equally both reporter constructs illustrates the specificity of the Ras-Endotag-siRNA#4 for the mutation. RAS-G12V/DNP53 transformed MEFs of Ccnd1−/− genotype were used for this experiment.

FIG. 10 represents a histogram showing the tumor burden dynamics of RAS-G12V/DNP53 transformed MEFs of Ccnd1−/− genotype rescued by the Tagged transgenes from FIG. 8. In vivo, TAG-RNAi illustrates the functional impact of the Ras-Endotag-siRNA#4 leading to the knock down of the CycD1 transgene fused to the G12V-Endotag (curve with circles) and to tumor growth arrest). No functional impact of Ras-Endotag-siRNA#4 is observed in tumors where the CycD1 transgene is fused to the KRAS-WT-Endotag (curve with triangles). Values are represented as average tumor size in mm³ of n=5 tumors+/− standard error of the mean. X-axis: days; y-axis: average tumor burden in mm³.

FIG. 11 represents an Immunoblot for KRAS (1.1 anti KRAS short exposure and 1.2 anti KRAS long exposure) using lysates from SW620 (KRAS-G12V mutated; i.) or HT29 (KRAS WildType; ii.) cell lines after treatment with Ras-Endotag-siRNA#4 (B and C) or irrelevant negative control HA-siRNA (A and D. Note the strong down-regulation of G12V-KRAS mutant in SW620 cell line by Ras-Endotag-siRNA#4, whereas only a marginal knock down is observed in wildtype KRAS HT29 cell line.

FIGS. 12-14—TAG-RNAi development using Ccnd1Ntag/Ntag and Ccnd1Ctag/Ctag MEFs

FIG. 12 represents a CycD1 Immunoblot (1.) of Ccnd1 Ctag/Ctag MEFs lysates after TAG-RNAi titration using an increasing final concentration of 0.1, 1 or 10 nM HA-siRNA (B) compared to 10 nM of Scramble siRNA (A). Load charge is evaluated with an anti-actin antibody (2)

FIG. 13 represents Ntag-CycD1 (left graph) and Ctag-CycD1 (right graph) mRNA relative quantification by RT-qPCR after TAG-RNAi (1: scramble; 2: Flag siRNA and 3: HA siRNA) in two different clones of Ccnd1Ntag/Ntag or Ccnd1Ctag/Ctag MEFs transformed by HRAS-G12V and Dominant Negative P53 (DNP53). The black box is the nucleotide sequence encoding for FLAG tag, the grey box is the nucleotide sequence encoding for HA tag. Error bars=SD, n=3.

FIG. 14 represents the relative Ntag-CycD1 (left graph) and Ctag-CycD1 (right graph) protein abundance, after TAG-RNAi (1: scramble; 2: Flag siRNA and 3: HA siRNA) in respectively two clones (A, B) of MEFs of Ccnd1Ntag/Ntag (A, B left graph) or Ccnd1Ctag/Ctag (A, B right graph) genotype, measured by Tandem-HTRF using FLAG antibody as a donor and ha, sc, abl and ab3 antibodies as acceptors (see methods section)1. Error bars=SD, n=3.

FIGS. 15-16—G1-Cyclins off-targeting by CycD1 siRNAs revealed thanks to TAG-RNA

FIG. 15 represents an immunoblot using anti-cyclin D1 (A) and actin (B) antibodies of lysates from RAS/DNP53 transformed MEFs of Ccnd1Ctag/Ctag (i) and Ccnd1+/+(ii) genotype after TAG-RNAi (Flag: 2; HA; 3) or RNAi against CycD1 using three different siRNAs (Nat: 4, Qia: 5 and Life: 6) and as control scramble siRNA (1).

FIG. 16 represents an immunoblot for G1-Cyclins by using anti-cyclin D2 (1), anti-cyclin D3 (3b), anti-cyclin E1 (4), and anti CDK4 (5) antibodies (and controlled with anti-actin antibody (6)) of lysates from RAS/DNP53 transformed MEFs of Ccnd1−/− genotype after Flag-RNAi as a control (A) or RNAi against CycD1 using three different siRNAs (Nat: B, Qia: C, Life: D). Note the down-regulations of 1-CycD2 protein after CycD1 RNAi using the Nat siRNA, 2-CycD3 protein after CycD1 RNAi using both Life and Qia siRNAs and 3-CycE1 protein after all CycD1 RNAi compared to control FLAG-RNAi.

FIGS. 17A-J—Comparative analysis of transcriptome profiles after CycD1-RNAi or TAG-RNAi

Venn diagrams illustrating the degree of overlap (both in total number and %) of genes differentially expressed between two siRNAs applied on RAS/DNP53 transformed MEFs of Ccnd1Ctag/Ctag genotype. The total number of genes differentially expressed after each siRNA treatment is1—Flag-siRNA: 862; 2—HA-siRNA: 2670, 3—Nat-siRNA: 448, 4—Life-siRNA: 1700, 5—Qia-siRNA: 604. A represents the transcription profile of Flag-RNAi versus HA-RNAi, B represents HA versus Nature, C is FLAG vs. Life, D is Quia vs. Life, E is HA vs. Quia, F is HA vs. Life, G is Nature vs. Life, H is Flag vs. Nat, I is Flag vs. Quia and J is Quia vs. Nat,

FIGS. 18-20—In vivo Comparison of CycD1-RNAi or TAG-RNA functional Incidence

FIG. 18 represents an immunoblot after TAG-RNAi (Flag siRNA: 1, HA siRNA: 2, scramble siRNA: 3) on RAS/DNP53 transformed MEFs of Ccnd1−/− genotype rescued by FLAG-HA-Tagged (i) or Untagged-CycD1 (ii) transgene. A: anti-cyclin D1 antibody and B: anti actin antibodies

FIG. 19 is a schematic representation of the conventional RNA interference approach using siRNAs designed to target a specific gene of interest in wildtype cells while no impact is expected in cells where this target has been genetically ablated. In this setting, the transcriptome and functional impact should be unaltered in the genetic knock out cells. E: Off targets; A: Phenotype ? Transcriptome ?; B: Identical phenotype, Identical transcriptome and C: Good control for Gene A siRNA off-target functional incidence.

FIG. 20 is a graph representing the in vivo RNAi functional impact on tumor burden dynamics of RAS-G12V/IDNP53 transformed MEFs of Ccnd1−/− genotype rescued by Tagged-CycD1 (curve with squares) or Untagged-CycD1 (curve with diamonds) transgene, or not (parental cells stably expressing GFP, curve with triangles). Note that CycD1-specific siRNAsinduce a tumor progression arrest of CycD1l-null tumors (curve with triangles) which is reversed after siRNA treatment arrest. In parallel, HA-siRNA was used in this experiment to demonstrate the specificity of TAG-RNAi (curves with squares for which tumor growth is inhibited or curve with diamonds where no effect is observed). RNAi treatment (illustrated by the bar) was initiated on the morning of Day 0 (see methods). Values are represented as average tumor size of n=6 tumors+/− standard error of the mean.

FIG. 21-23—TAG-RNA for the targeting of any gene in any cell type

FIG. 21 represents an immunoblot of mouse 3T3 (i) or human MCF7 cell lines (ii) expressing FLAG-HA-CycD1 transgene w/wo TAG-RNAi treatment (Flag (2) or HA (3)), and compared to the scramble siRNA treatment (1). Protein are revealed with anti-cyclin D1 (A) and actin (B) antibodies.

FIG. 22 represents the knock down efficiency of transgenic Flag-mCherry-HA (left panel) or Flag-CycD1-HA expression (right panel) in wildtype RAS/DNP53 transformed MEFs measured by RT-qPCR after TAG-RNAi using Flag-siRNA (2) or HA-siRNA (3) vs scramble siRNA (1). Error bars=SD, n=3.

FIG. 23 represents an immunoblot from lysates of Ccnd1Ctag/+ MEFs expressing the CycD1 mRNA depicted on the right schematic (tagged and untagged). Note that only the tagged version of CycD1 (upper band) is affected by TAG-RNAi treatment and not the WT untagged version of CycD1 (lower band) when labelled with anti-cyclin D1 antibody (A). B: labelling with anti-HA antibody; C: labelling with anti-actin antibody.

FIG. 24-28—In vivo TAG-RNA versatility for the study of tumor growth dynamics after reversible gene knock down

FIG. 24 represents an immunoblot of tagged or untagged WT-CycD1 or T286A-CycD1 mutant (which is hyper-stable and oncogenic), expressed within the same Ccnd1−/− MEFs cell line. Note that only the tagged version (whether WT or mutated on T286) is sensitive to TAG-RNAi treatment (**, lane 6 to 9). 1: TAG-CycD1+T286A; 2: TAG-T286A+CycD1; 3: TAG-T286A; 4: TAG-CycD1; 5: CD1−/− (parental); 6: TAG-CycD1; 7: TAG-T286A; 8: TAG-T286A+CycD1 and 9: TAG-CycD1+T286A. #1: clone 1; #2 clone 2. *: treatment with siRNA. A1 and A2: band of cyclin D1. B: actin.

FIG. 25 represents a graph showing the tagged-T286A-CycD1 transgene driven tumor progression analysis w/wo HA-siRNA (curve with squares), FLAG-siRNA (curve with triangles) or Scramble-siRNA (curve with diamonds) (illustrated by the bars). Note the versatility of the approach with tumors relapsing after TAG-RNAi treatment pause, but remaining sensitive to the treatment when applied again later. Values represent the average tumor size of n=10 tumors+/− standard error of the mean. Ccnd1+/+3T3 cells were used for this experiment. Y-axis: Average tumor size (mm³) and x-axis: days.

FIG. 26 represents a graph showing T286A-CycD1 (curve with diamonds) or Tagged-T286A-CycD1 (curve with squares) driven tumor progression analysis w/wo in vivo TAG-RNAi. Note the specificity of the TAG-RNAi approach which specifically impinges on Tagged-T286A-CycD1-driven tumor progression on one flank of the mouse (squares) but has no off-target impact on untagged T286A-CycD1-driven tumor growth on the other flank of the same animal (diamonds). Values are represented as average tumor size of n=10 tumors+/− standard error of the mean. Ccnd1+/+3T3 cells and FLAG-siRNAwere used in this experiment. Curve with triangle represents T286A-CycD1 treated with control scramble siRNA. Y-axis: Average tumor size (mm³) and x-axis: days.

FIG. 27 represents a histogram of 5 days Tumor growth index (size of the tumor/size of the tumor 5 days before) of Ccnd1+/+3T3 cells expressing Tagged-T286A-CycD1 (4-8) or untagged T286-CycD1 (1-3) after in vivo TAG-RNAi. Note the equal efficiency of FLAG or HA-RNAi, but no significant additive effect of FLAG then HA RNA interference on tumor burden (see methods). Results are represented as average values of n=10 tumors+/− standard deviation, where the size of each tumor is measured and divided by its size 5 days before. 1: Flag-siRNA; 2: FLAG-siRNA+HA-siRNA; 3: HA-siRNA; 4: HA-siRNA; 5: FLAG-siRNA+HA-siRNA; 6: FLAG-siRNA; 7: SCR-siRNA and 8: vehicle. X-axis: days. *p<0.05, **p<0.01; pairwise comparison using two-tailed paired (brown bars versus blue bars) or unpaired (blue bars versus blue bars) Student's t test.

FIG. 28 represents a graph showing the tumor growth dynamics upon TAG-RNAi treatment, followed by treatment pause (represented by slashes preceding the relapse of the tumors on the graph), followed by TAG-RNAi treatment inversion, illustrating the various possibilities of the versatile TAG-RNAi approach. Measures of the tumor size are done in the morning just before TAG-RNAi treatment, and day 1 is the first day of treatment. Values are represented as average tumor size of n=10 tumors+1-standard error of the mean. Curve with diamonds: HA-siRNA/Vehicle; curve with squares: FLAG-siRNA/SCR-siRNA; curve with triangles: SCR-siRNA/FLAG-siRNA and curve with circles: Vehicle/HA-siRNA. Y-axis: Average tumor size in mm³ and x-axis: days.

FIGS. 29-33—In vivo TAG-RNAi applied to the murine HRAS-G12V oncogene

FIG. 29 is a schematic representation of the implantation of murine “Tagged-HRAS-G12V” expressing cancer cells on one flank of immune compromised mice (black circle) whereas murine “untagged-HRAS-G12V” control cells are implanted on the contralateral flank (grey circle). Cells from the black circle can be targeted by the siRNA specific to the genetic TAG whereas cells from the grey circle are insensitive to this siRNA. The cancer cells were generated using MEFs transformed by the SV40 Large T and the human HRAS-G12V transgenes.

FIG. 30 represents an immunoblot using lysates from murine RAS-G12V/Large T transformed MEFs that express RAS-G12V protein from a transgene that is fused (left immunoblot) or not (right immunoblot) to genetic sequences of Flag, schematized as a black box (in 5′ before the translation initiation Kozak sequence) and HA, schematized as a grey box, (in 3′ after the stop codon) localized in the untranslated region of the transgenic mRNA. Note that only the transgene carrying the Flag and HA sequences can be silenced by Flag or HA-specific siRNA. I: anti RAS antibody and ii: anti actin antibody. 1: untransformed cells; 2: cells treated with scramble siRNA, 3; cells treated with FLAG-siRNA and 4: cells treated with HA-siRNA. Grey box with K: schematic representation of Kozak sequence; white box with R: schematic representation of murine RAS-G12V cDNA. * represents a stop codon. +++: tumor

FIG. 31 is a graph showing Tagged-HRAS-G12V driven tumor progression w/wo in vivo TAG-RNAi using HA-siRNA or Scramble-siRNA. Note that the “Tagged” tumor progression is decreased after TAG-RNAi. Values are represented as average tumor size of n=10 tumors+/− standard error of the mean. Ccnd1+/+3T3 cells were used in this experiment. Curve with diamonds: HA-siRNA; curve with squares: scramble siRNA. Y-axis: tumor volume (mm³) and y-axis: days.

FIG. 32 is a graph showing the in vivo growth kinetics of Tagged-HRAS-G12V-driven tumors after TAG-RNAi. The size of each tumor is measured (Day 9 of graph FIG. 31) and divided by its size 5 days before (Day 5 of graph FIG. 31). Results are represented as average values +/− standard deviation from two independent experiments performed with two independent biological clones, each experiment comprising n=5 tumors per clone. A: scramble siRNA; B: HA-siRNA.

FIG. 33 is a graph showing the untagged-HRAS-G12V driven tumor progression w/wo in vivo RNAi using HA-siRNA or Scramble-siRNA. Note the absence of significant impact on tumor progression with both Scramble and HA-siRNA. Values are represented as average tumor size of n=10 tumors+/− standard error of the mean. Curve with squares: HA-siRNA; curve with diamonds: scramble siRNA. *p<0.05; ***p<0.001; pairwise comparison USING two-tailed unpaired Student's t test (FIGS. 32 and 33).

FIGS. 34-36—Rapid TAG-RNA screening using 386-well plate Tandem-HTRF readouts

FIG. 34 represents the relative Ntag-CycD1 protein abundance measured by Tandem-HTRF using FLAG as a Förster Resonance Energy Transfer “donor” antibody and ha, abl, ab3 and sc antibodies as “acceptors” for the screening of the V5 Tag-specific siRNAs (see Table 1). On the bottom schematic is represented the nucleotide sequence encoding for the V5 Tag (separated right grey box) and which corresponds to the peptide from 95 to 108 (GKPIPNPLLGLDST SEQ ID NO: 51) of RNA polymerase a subunit of simian parainfluenza virus type 5, but that has been fused to the non-coding region of the reporter transgene encoding for Ntag-CycD1. The asterisk illustrates the Stop codon of the transgene. Error bars=SD, n=3. 1: Scr-siRNA, 2: V5-siRNA1, 3: V5-siRNA2, 4: V5-siRNA4, 5: V5-siRNA5, 6: V5-siRNA6, 7: V5-siRNA7, 8: V5-siRNA8, 9: V5-siRNA9, 10: V5-siRNA10, 11: V5-siRNA11, 12: V5-siRNA12, 13: V5-siRNA13, 14: V5-siRNA14, 15: V5-siRNA15, 16: V5-siRNA16, 17: V5-siRNA17, 18: V5-siRNA18, 19: V5-siRNA19, 20: V5-siRNA20, 21: V5-siRNA21, 22: V5-siRNA22 and 23: FLAG-siRNA.

FIG. 35 represents the relative MYC-CDK4-V5 fusion protein abundance measured by Tandem-HTRF using MYC as a Förster Resonance Energy Transfer “donor” antibody and v5 antibody as an “acceptors” for the screening confirmation of V5 Tag-specific siRNAs compared to a (see Table 1). The nucleotide sequence encoding for the V5 Tag has been fused to the coding region of the reporter transgene encoding for CDK4 in this experiment. Error bars=SD, n=3. 1: Scr-siRNA, 2: V5-siRNA1, 3: V5-siRNA2, 4: V5-siRNA4, 5: V5-siRNA5, 6: V5-siRNA6, 7: V5-siRNA7, 8: V5-siRNA8, 9: V5-siRNA9, 10: V5-siRNA10, 11: V5-siRNA11, 12: V5-siRNA12, 13: V5-siRNA13, 14: V5-siRNA14, 15: V5-siRNA15, 16: V5-siRNA16, 17: V5-siRNA17, 18: V5-siRNA18, 19: V5-siRNA19, 20: V5-siRNA20, 21: V5-siRNA21 and 22: V5-siRNA22.

FIG. 36 represents the impact of mutations in the coding sequence of FLAG or HA peptides on the knock down efficiency by TAG-siRNAs (see table 1). The right schematic illustrates the mismatches (star for a match versus exclamation mark for a mismatch) that reside between the targeted sequence and the siRNA used. Note that due to the mismatches, FlagN-siRNA (i.) no longer inhibits Flag-CycD1-HA transgene and is less efficient than FlagC-siRNA (ii.) for the inhibition of Ctag-CycD1 transgene. Scramble-siRNA was used as a negative control for the basal level of each transgenic construct expression. Flag-siRNA and HA-siRNA were used as positive interfering RNAs working on all transgenic constructs. Y-axis: Relative protein abundance (%). 1: scramble siRNA; 2: Flag-siRNA; 3: HA-siRNA; 4: FlagC-siRNA and 5: FlagN-siRNA.

FIGS. 37-40—TAG-RNA applied to the endogenous Ras-G12V genetic mutant tag

FIG. 37 represents the relative Ntag-CycD1 reporter protein abundance measured by Tandem-HTRF using FLAG as a Förster Resonance Energy Transfer “donor” antibody and ha, abl, ab3 and sc antibodies as “acceptors” for the screening of Ras-G12V Endotag-specific siRNAs (see Table 1). Error bars=SD, n=3. 1: Scr-siRNA: 2: Flag-siRNA: 3: Ras-G12V-Endotag-siRNA1, 4: Ras-G12V-Endotag-siRNA2, 5: Ras-G12V-Endotag-siRNA3, 6: Ras-G12V-Endotag-siRNA4, 7: Ras-G12V-Endotag-siRNA5, 8: Ras-G12V-Endotag-siRNA6, 9: Ras-G12V-Endotag-siRNA7, 10: Ras-G12V-Endotag-siRNA8, 11: Ras-G12V-Endotag-siRNA9, 12: Ras-G12V-Endotag-siRNA10, 13: Ras-G12V-Endotag-siRNA11, 14: Ras-G12V-Endotag-siRNA12, 15: Ras-G12V-Endotag-siRNA13, 16: Ras-G12V-Endotag-siRNA14, 17: Ras-G12V-Endotag-siRNA15, 18: Ras-G12V-Endotag-siRNA16, 19: Ras-G12V-Endotag-siRNA17, 20: Ras-G12V-Endotag-siRNA18, 21: Ras-G12V-Endotag-siRNA19, 22: Ras-G12V-Endotag-siRNA20 and 23: Ras-G12V-Endotag-siRNA21.

Black columns: CycD1-STOP-RASWT Tag constructions, Dark grey columns: CycD1-STOP-RASG12V Tag constructions

FIG. 38 represents an immunoblot of HRAS-G12V/DNP53 transformed MEFs of Ccnd1−/− genotype rescued by the CycD1 Tagged transgenes (WT-Endotag (ii) or G12V-Endotag (i)) and targeted by TAG-RNAi using human Kras-G12V-Endotag-specific siRNA#4 (2), #5 (3), or #16 (4) and scramble (1), which were showing the most promising mutation specific knock down from the screening performed in FIG. 37. A: anti-cyclin D1; B: anti-actin.

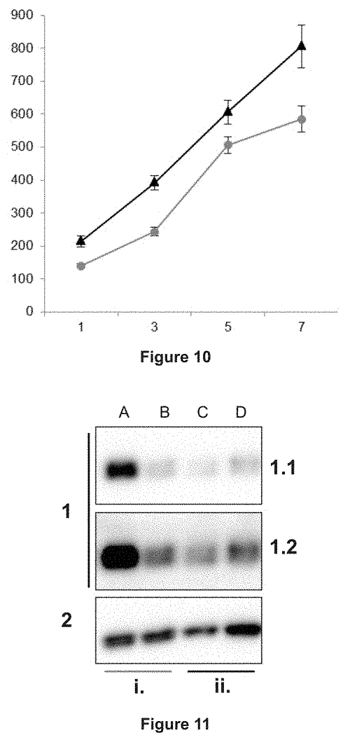

FIG. 39 represents a histogram showing the in vivo tumor growth kinetics of HRAS-G12V/DNP53 transformed MEFs of Ccnd1−/− genotype rescued by the CycD1 Tagged transgenes (WT-Endotag or G12V-Endotag) and targeted by TAG-RNAi using human Kras-G12V-Endotag-specific siRNA#4 or HA-siRNA. The size of each tumor from FIG. 10 is measured before and after treatment and divided by its size 2 days before (Day 5/Day 3 before treatment in black bars and Day 7/Day 5 after treatment in grey bars). Results are represented as average values+/− standard deviation with n=5 tumors. *p<0.05; ***p<0.001; pairwise comparison USING two-tailed unpaired Student's t test. A: G12 V-Endotag/HA-siRNA; B: WT-Endotag/siRNA#4, C: G12V-Endotag/siRNA#4.

FIG. 40 represents an immunoblot for CycD1 (A) (and control actin (B)) using lysates from HT29 (i) and SW620 (ii) human cancer cell lines after treatment with CycD1-siRNA (1) or irrelevant negative control HA-siRNA (2). Note the strong down-regulation of CycD1 expression attesting for good siRNA transfection efficiency in both cell lines.

FIG. 41 is a schematic representing the generation of a TAG-RNAi strategy specific to the BRaf mutation (V600E-Endotag). The mutant V600E-Endotag (black) or the non-mutated WT-Endotag (grey) sequence spans from the −20 to the +20 nucleotides around the mutation and are fused to the non-coding part of the reporter gene encoding for CycD1.

FIG. 42 represents the relative CycD1 reporter protein abundance measured by Tandem-HTRF using SC450 as a Firster Resonance Energy Transfer “donor” antibody and abl and ab3 antibodies as “acceptors” for the screening of BRAF-V600E Endotag-specific siRNAs (see Table 1). Error bars=SD, n=3. 1: Scr-siRNA: 2: HA-siRNA: 3: Raf-V600E-Endotag-siRNA1, 4: Raf-V600E-Endotag-siRNA2, 5: Raf-V600E-Endotag-siRNA3, 6: Raf-V600E-Endotag-siRNA4, 7: Raf-V600E-Endotag-siRNA5, 8: Raf-V600E-Endotag-siRNA6, 9: Raf-V600E-Endotag-siRNA7, 10: Raf-V600E-Endotag-siRNA8, 11: Raf-V600E-Endotag-siRNA9, 12: Raf-V600E-Endotag-siRNA10, 13: Raf-V600E-Endotag-siRNA11, 14: Raf-V600E-Endotag-siRNA12, 15: Raf-V600E-Endotag-siRNA13, 16: Raf-V600E-Endotag-siRNA14, 17: Raf-V600E-Endotag-siRNA15, 18: Raf-V600E-Endotag-siRNA16, 19: Raf-V600E-Endotag-siRNA17, 20: Raf-V600E-Endotag-siRNA18, 21: Raf-V600E-Endotag-siRNA19, 22: Raf-V600E-Endotag-siRNA20 and 23: Raf-V600E-Endotag-siRNA21.

Dark grey columns: CycD1-STOP-BRAFWT Tag constructions, Black columns: CycD1-STOP-BRAF-V600E Tag constructions

### EXAMPLE

The inventors reasoned that ectopic RNAi could rely on a tag sequence linked to a specific locus of interest to be targeted. The idea behind using a tag complementary to the siRNA sequence, but absent from control cells, is that cells without the tag would correspond to scrambled controls in a classical siRNA experiment and also to rescued control cells. With the TAG-RNAi alternative, control cells are exposed to the exact same siRNA molecule than the responding cells to be challenged. This approach can theoretically unmask phenotypic alterations that arise with “off-target” interference. As a consequence, TAG-RNAi provides an accurate functional signature to fairly compare with gene ablation phenotypes.

To demonstrate our hypothesis, the inventors took advantage of genetically engineered mice expressing FLAG-HA tagged versions of Cyclin D1 (CycD1) at physiological levels. These strains produce functional N-terminal (Ntag-CycD1) or C-terminal (Ctag-CycD1) Flag-HA tagged-CycD1 protein. Hence, FLAG or HA RNA interference will be blind to wildtype Ccnd1 gene expression but interfere with Tagged-CycD1 mRNA translation (FIG. 1).

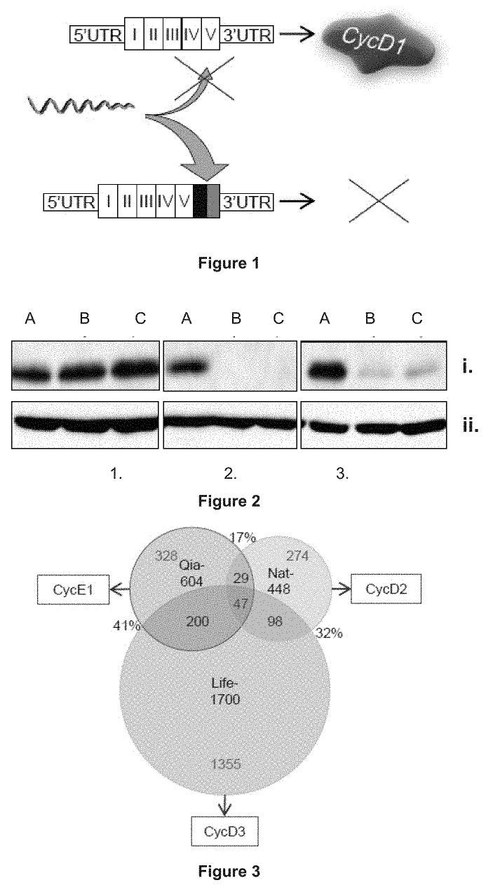

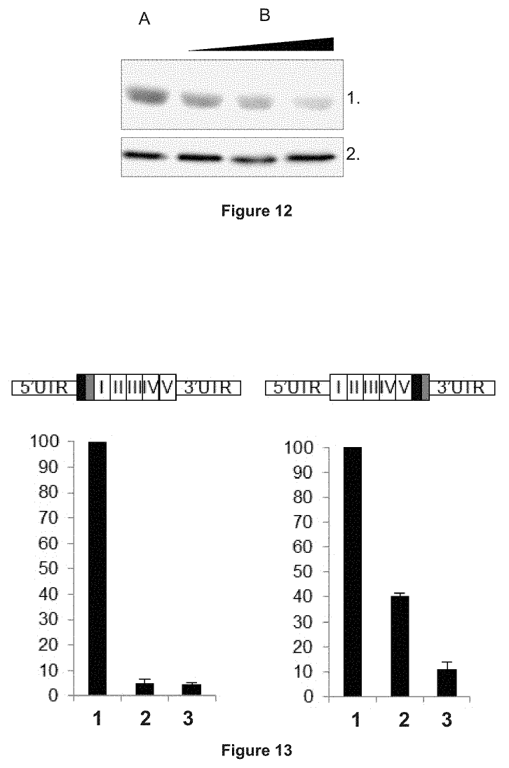

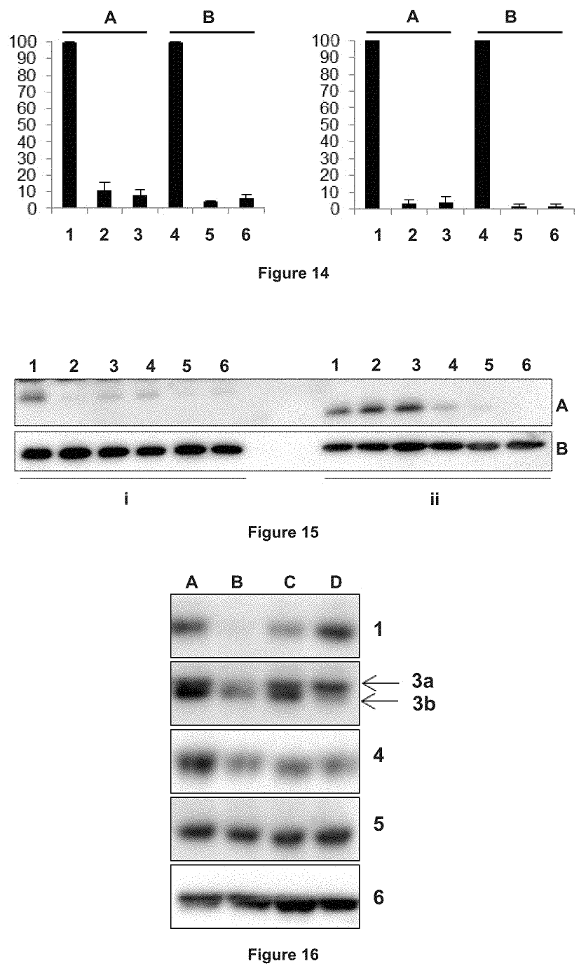

The inventors first isolated Flag or HA siRNAs specific to knock down Tagged-CycD1 in a dose-dependent manner, whether the target mRNA sequence is at the 5′ or at the 3′ end of the mature messenger RNA (FIG. 2, FIG. 12 and FIG. 14, Table 1).

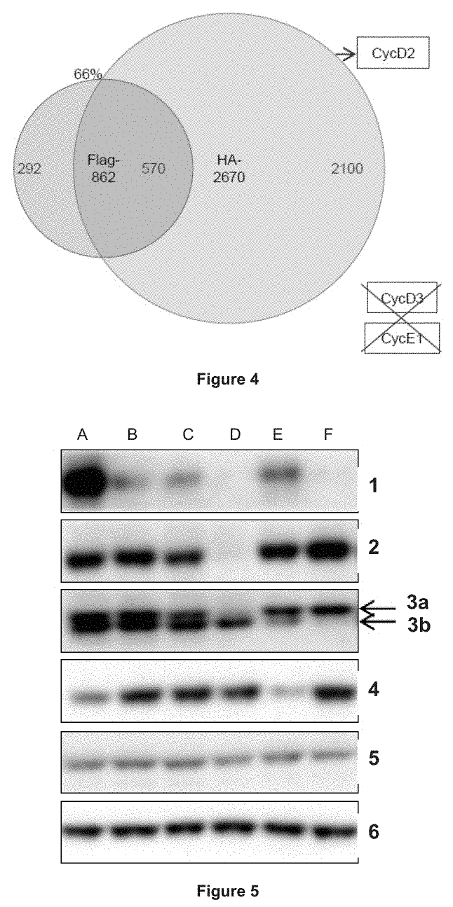

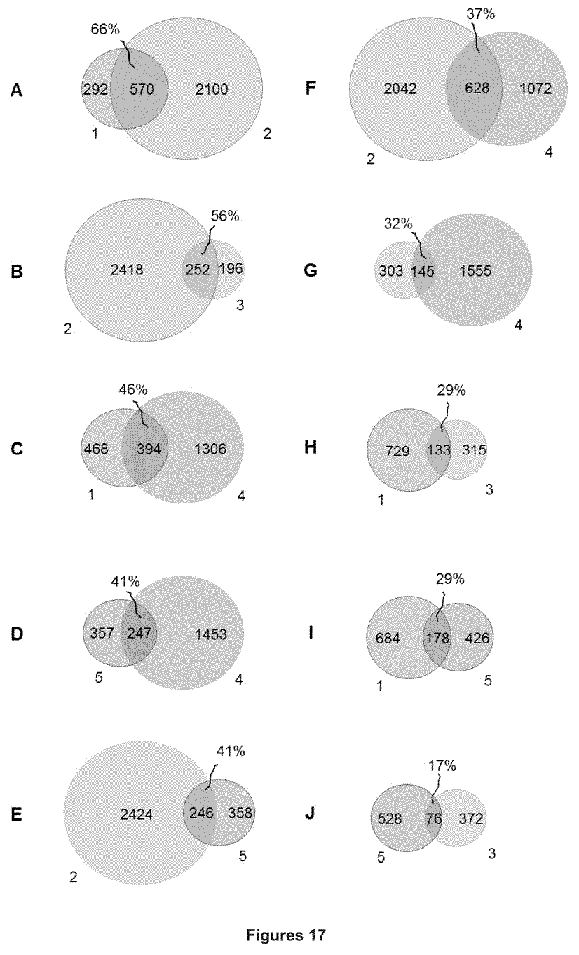

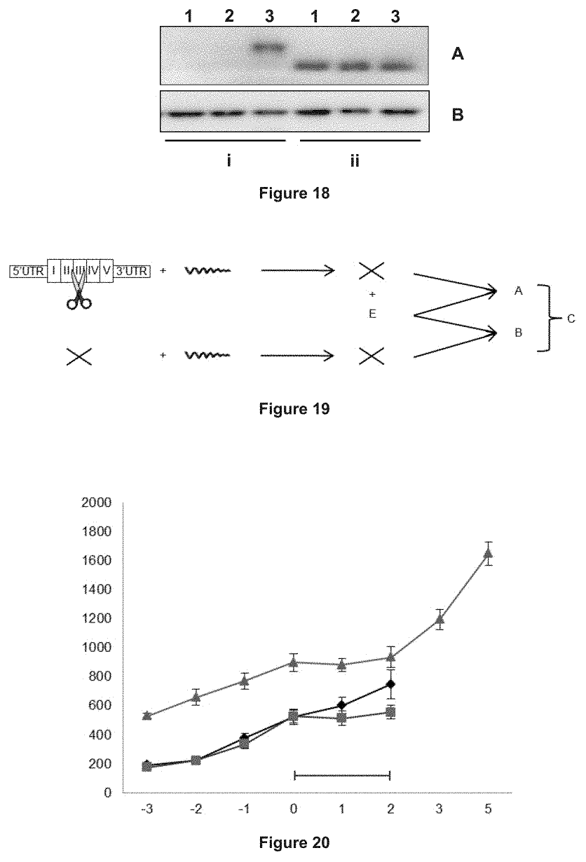

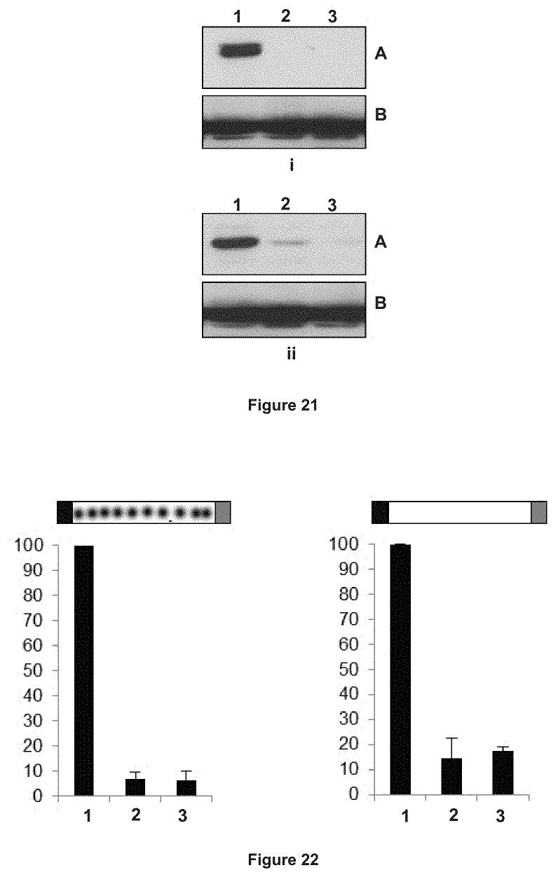

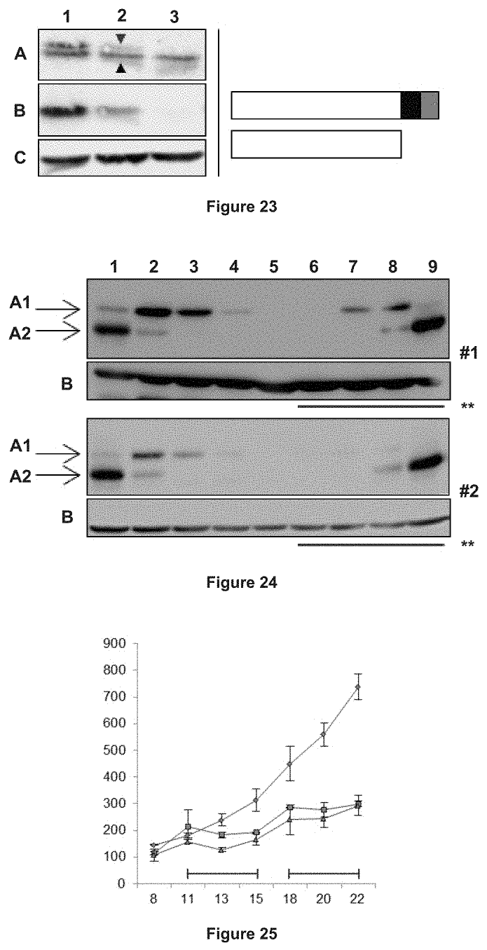

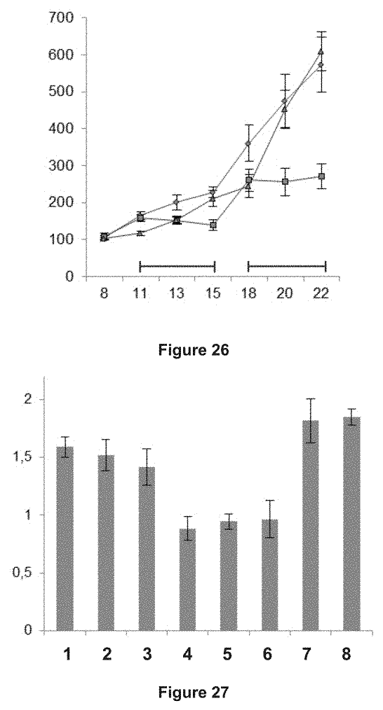

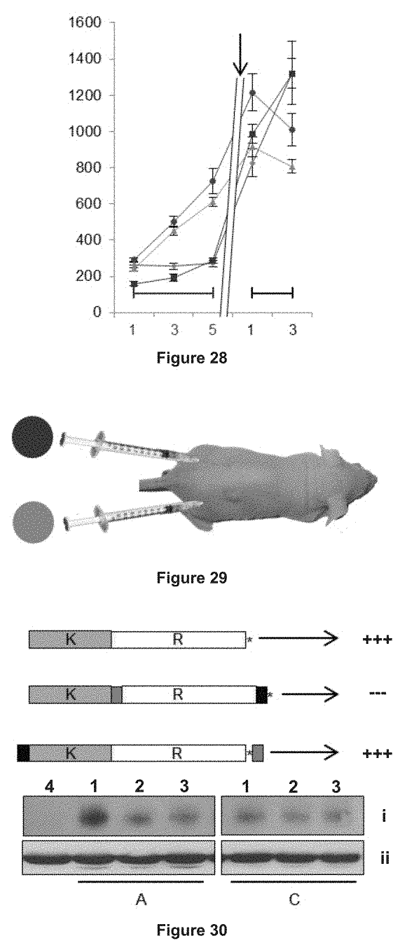

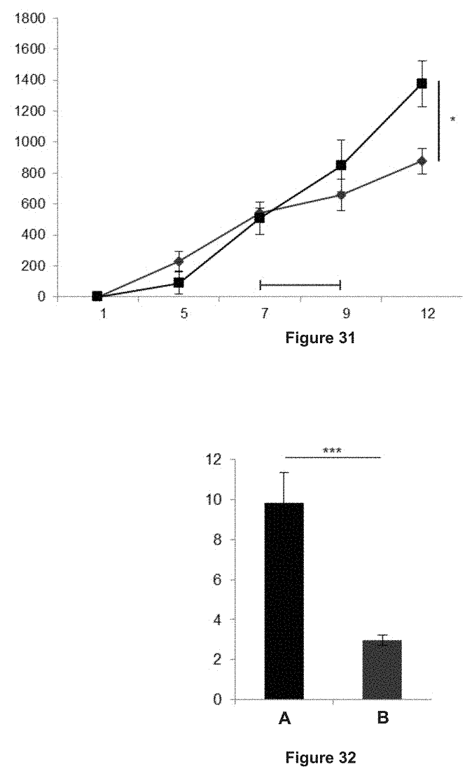

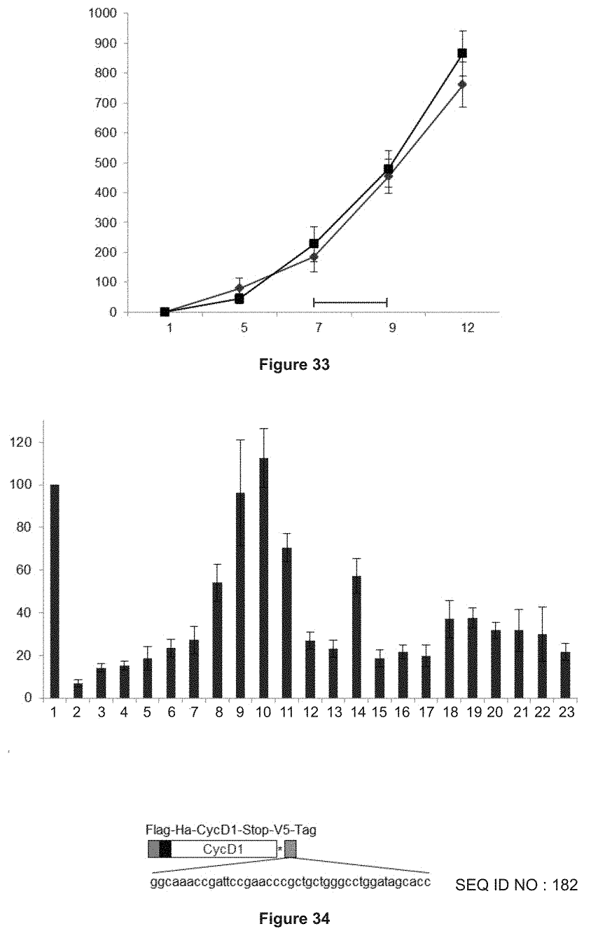

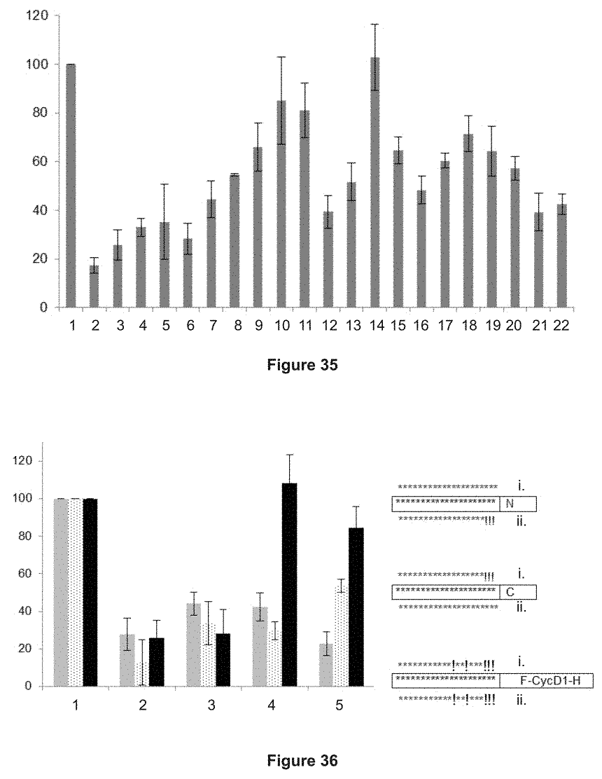

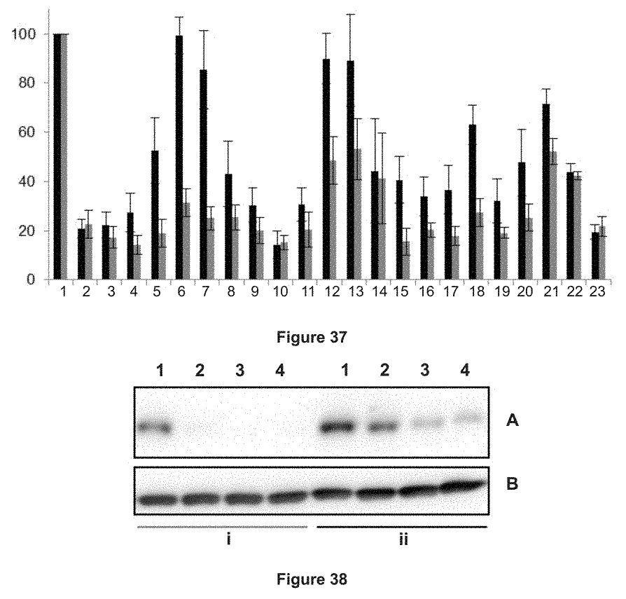

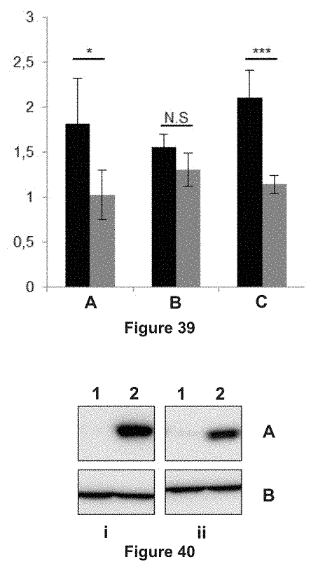

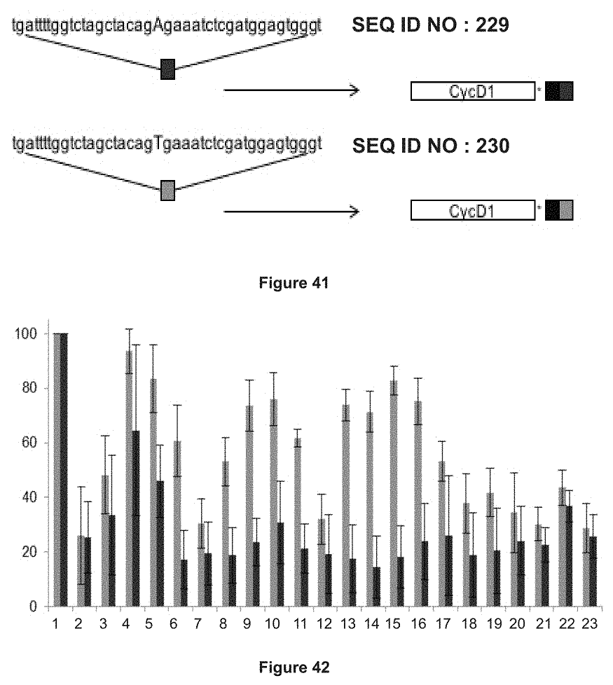

Then, to test the specificity and the efficiency of TAG-RNAi compared to three independent siRNAs, the inventors performed RNA-Sequencing experiments on Mouse Embryonic Fibroblasts (MEFs) transformed by the HRAS oncogene and Dominant Negative P53 (DNP53). Following RNAi using either siFLAG, siHA, a published siRNA against CycD1, or two different commercial siRNA sequences against CycD1, the inventors collected the expression profiles of cells expressing Ctag-CycD1. As expected, Ctag-CycD1 mRNA was knocked down with all five siRNAs tested (FIG. 15). However, the global transcription profile deviates less between TAG-siRNAs than between CycD1-siRNAs (FIG. 4, FIG. 5, FIG. 17). More surprisingly, close inspection of genes only differentially expressed after CycD1 RNA interference but not TAG-RNAi, revealed the down-regulation of other G1-Cyclins, like Cyclin D3 and Cyclin E1 (FIG. 3-5). It is well established that all G1-Cyclins belong to the same functional group and promote cell cycle and tumor progression. Our results show unexpectedly that the targeting of CycD1-null cells by siRNAs supposed to be specific to CycD1, leads to the down-regulation of other G1-Cyclins too (FIG. 16). This suggests that a functional off-targeting by three different ectopic siRNAs against CycD1 could alter fundamental properties of these CycD1-null cancer cells. In contrast, TAG-RNAi technology provides functional observations that can confidently be attributed to the specific targeting of the tagged gene of interest.

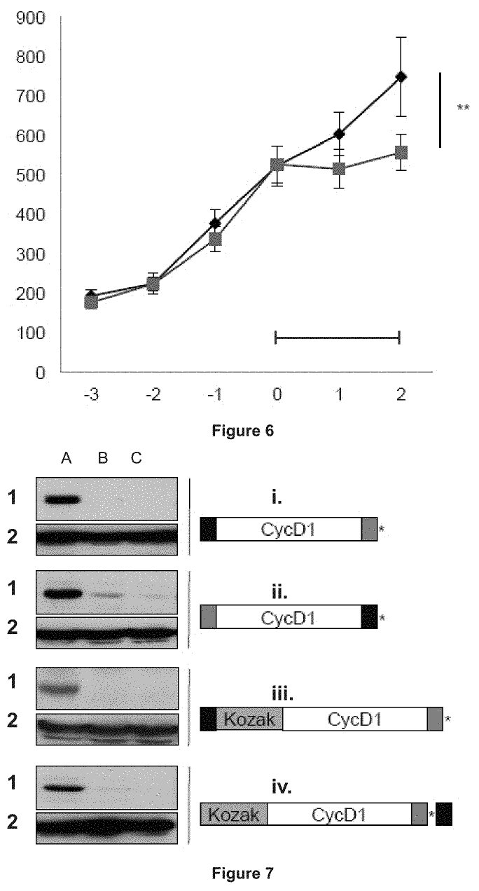

Furthermore, TAG-RNAi offers in vivo an opportunity for the functional exploration intrinsic to the targeted cells. The strength of the approach relies on the biological response of “tagged tumors” on one flank of the recipient mouse, while no impact is expected on “untagged tumor” of the other flank of the same animal. Indeed the targeting of Tagged-CycD1 which induced tumor growth inhibition, as reported by conditional genetic ablation, demonstrated this (FIG. 6, FIG. 18). Surprisingly, whereas tumors expressing untagged-CycD1 remain unaffected by TAG-RNAi, a striking phenotype characterized by tumor progression arrest is induced in CycD1-null cancer cells treated with “CycD1-specific” siRNAs (FIG. 19 and FIG. 20). Such in vivo experimental artifact strongly suggest that “specific” CycD1 siRNAs exert an “off-target” functional pressure on tumors and should be used with caution to investigate the impact of CycD1 on cancer development. Besides, this off-target induced phenotype would not be revealed when performing parallel experiments using the usual Scramble siRNA control. TAG-RNAi on the other hand rules out the risk of biological misinterpretation following in vivo gene knock down by RNA interference.

Using the same siRNA molecule, TAG-RNAi is applicable in any cell type and for the targeting of any (single or multiple) tagged transgene(s) (FIG. 21 and FIG. 22). Thanks to heterozygous knock in strains, TAG-RNAi also allows the specific silencing of the product of one tagged allele while sparing the other wildtype (untagged) allele (FIG. 23). Thus, it is for example technically possible to co-express in the same cell, one mutant version that can be targeted by TAG-RNAi, and an additional wildtype untagged version for which the expression is unchanged, or vice versa (FIG. 24). Therefore, In vitro and in vivo, TAG-RNAi offers a wide range of opportunities to study the functional dynamics of transient knock down of any gene of interest (FIG. 25-27).

The targeting of any mRNA sequence can be achieved by TAG-RNAi conducted inside or outside of the translated region (FIG. 7). This useful alternative avoids peptide sequence modification of the candidate protein to be targeted and prevents the risk of subsequent loss-of-function. Tags added to the non-coding region of the Hras mRNA allows to target this untagged oncogene in vivo using TAG-RNAi (FIG. 31-33).

In a siRNA screening perspective, the inventors show using the V5 tag, that the isolation of specific siRNAs for any tag is relatively easy (FIG. 34). Additionally, while keeping a constant peptidic tag sequence, one can design mutations rendering Tagged-mRNA resistant to RNAi (FIG. 36).

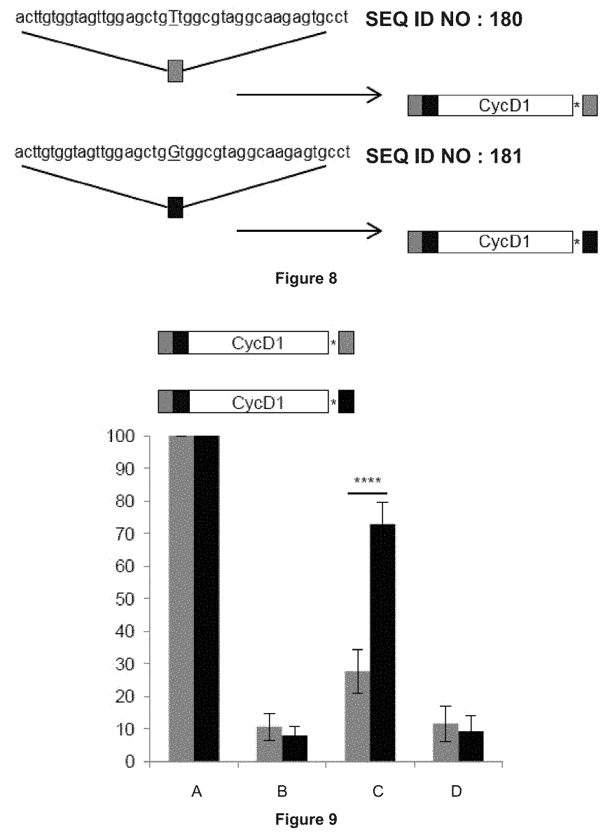

For this reason, in the frame of human therapeutics, the inventors wanted to explore endogenous mutant genetic tags (Endotags) that could be targeted specifically by TAG-RNAi in native cells. Many diseases are linked to genetic mutations and silencing such alterations while sparing wildtype “healthy” version of the candidate target could be a specific means of targeting only mutated sick cells in a clinical assay. Consequently, the inventors focused on a known 35 G>T alteration of the oncogene Kras which occurs at the level of the codon 12 and changes the amino acid sequence from a Glycine to a Valine in human cancers. By extracting the 20 nucleotides upstream and downstream of this mutation the inventors generated the so-called G12V-Endotag (FIG. 8). As a control, the inventors used the same 40 nucleotides from the wildtype version of Kras and named this the WT-Endotag (FIG. 8). Moving along the G12V-Endotag sequence base by base, the inventors screened all 21 possible siRNAs that could potentially silence the reporter mRNA encoding for CycD1 and carrying the G12V-Endotag, to induce a minor effect on the reporter mRNA carrying the WT-Endotag (FIG. 37). From all the siRNA tested, G12V-Endotag-siRNA number 4 did knock down the reporter gene fused to G12V-Endotag but affected at the margin the reporter construct fused to the WT-Endotag (FIG. 9, FIG. 37 and FIG. 38). To probe for potential off-target side effects of this most promising “G12V-specific” siRNA#4 in vivo, the inventors investigated its impact on CycD1l-driven tumor growth like the inventors did earlier (FIG. 6). #4 appeared to be efficient in targeting the CycD1 transgene carrying the G12V-Endotag to repress tumor growth, while having no significant biological impact on tumors carrying the WT-Endotag (FIG. 10, FIG. 39). Finally, by testing in parallel its efficiency in SW620 (KRAS-G12V mutated) and HT29 (KRAS wildtype) human colorectal cancer cell lines, the inventors confirmed that G12V-Endotag-siRNA#4 specifically knocks down the G12V mutation of Kras oncogene but presents a limited incidence on wildtype human KRAS (FIG. 11, FIG. 40).

Then the inventors performed the same kind of screening approach applied to another well-known genetic hit leading to the generation of the BRAF-V600E mutated protein. This strongly oncogenic 1799T>A genetic event on the gene coding for BRAF is associated with severe morbidity and resistance to modem anti-cancer therapies using monoclonal antibodies like Cetuximab. Like the inventors did for KRAS-G12V, they extracted the 20 nucleotides upstream and downstream of the BRAF-V600E mutation to generate the so-called BRAFV600E-Endotag (FIG. 41). Using Tandem-HTRF against CycD1 and following the targeting of BRAFV600E-Endotag by RNAi we isolated several siRNAs (#11 to #14) able to silence the CycD1 reporter gene containing this tag but not the reporter gene containing the BRAFWT-Endotag (FIG. 42). Since this mutation severely cripples the therapeutic response of colorectal cancer patients, the inventors tested these siRNAs in BRAF-V600E mutated HT-29 human colorectal cancer cell line in clonogenic assays. The inventors found that the siRNA#11 can strongly impair the viability of HT-29 cells even in absence of any chemotherapeutic stress (data not shown).

Thus, the screening of endogenous mutant genetic tags may provide a fruitful way of delivering novel and specific endogenous TAG-siRNAs to test their reliability in non-mutated cells in vitro and in vivo.

To date and despite sophisticated algorithm-based rationale design, siRNA selectivity remains difficult to evaluate8. However, the inventors have shown that TAG-RNA interference is an efficient way for acquiring high confidence functional genomics signatures. TAG-RNAi provides a novel elegant and robust approach to alter specific gene expression, without carrying over functional side-effects. The unique versatility of TAG-RNAi can be declined for any gene of interest, by using simple tagged-transgenic constructs, or by the specific editing of endogenous genomic locus using technologies like CRISPR-Cas9 for instance. Finally, the use of pathogenic mutations can provide a unique opportunity to search for disease-specific TAG-siRNAs and to rapidly test their pre-clinical safety. Ultimately, TAG-RNAi could perhaps be amenable to therapeutic perspectives by lowering off-target downsides for the safer use of RNAi in clinics.

Rationale for TAG-RNA Development

RNA interference represents a strong potential therapeutic support to treat cancer. CycD1 is known to participate in cancer development. As a probe for potential therapeutic intervention by RNA interference and to ensure the unique knock down of CycD1, we ought to exclude “false-positive” phenotypic changes that could mislead our clinical strategy goal. By targeting Ctag-CycD1 and Ntag-CycD1 using FLAG or HA-siRNAs, we realized that no clear alteration of other G1-Cyclins was observed, contrary to the use of conventional CycD1-siRNAs. This led us to reconsider our view that CycD1 targeting by RNAi might be sufficient to prevent cell cycle in RAS-transformed cancer cells. It also alerted us on the dangerous scientific conclusion that could arise from such an experimental artifact related to the additional targeting of other G1-Cyclins by conventional siRNAs. That is why we decided to develop TAG-RNAi to gain confidence in the proper transcriptional and phenotypic signature of targeted cells. This way, any pharmacological intervention following RNAi screenings should have higher chances of success.

D-type Cyclins expression profile after conventional CycD1-RNAi or TAG-RNAi

Because G1-Cyclins levels of Ctag-CycD1 expressing cells seemed altered by CycD1-RNAi and not TAG-RNAi, we decided to explore the entire expression profile of these cells by RNA-Sequencing. It appears that CycD3 and CycE1 are only differentially expressed using one out of five siRNAs targeting CycD1. Considering that each of the five siRNAs tested (three raised against CycD1 and two raised against the FLAG-HA Tag), efficiently knocked down the expression of CycD1, we considered unlikely that this result would relate to the targeting efficacy of CycD1 between each siRNA. By testing these siRNAs in CycD1-null cells, we confirmed our prediction that they would alter other G1-Cyclins expression as an unspecific side-effect. We did not functionally explore whether this off-targeting is direct or indirect, but the simple alignment of the siRNAs tested with the cDNA of each G1-Cyclins reveals potential hybridization regions for the siRNAs we used. Concerning CycD2, we found that Nat-siRNA, life-siRNA and HA-siRNA lead to its down-regulation but not FLAG-siRNA. Hence, we remain sceptical about CycD2 expression being truly regulated by upstream CycD1 in Ras-transformed cells. In particular, Nat-siRNA also decreases CycD2 expression in CycD1-null cells.

Endotag-RNAi as a Clinical Intervention Perspective

Although TAG-RNAi appears as a reliable way for specifically targeting any gene of interest, its use for genetically unmodified primary human cells is still limited. To undertake functional studies in cellular models of human diseases, we believe that “natural” genetic mutations or SNPs may provide a powerful support for the development of base-specific Endotag-RNAi. Despite questionable mRNA off-targeting compared to TAG-RNAi based on 21 nucleotides rare genetic sequences, Endotag-RNAi provides at least a way to ensure the functional relevance of the targeted endogenous gene by using the same siRNA in cells that would not bare this mutation. Again, the benefit of this approach is to limit artificial phenotypes that would not only relate to the primary gene on-targeting but rather be the sum of multiple on and off-targeting consequences genome-wide. The subtraction of the off-targeting bias, at least at the functional level if not at the genome-wide transcription profile level, will certainly help to unmask true novel pharmacological targets and discard many false candidates for future therapeutics development. In addition the safety of such Endotag-siRNA can be easily tested using in cellulo viability models on healthy cells. For cancer therapeutic perspectives, any promising Endotag-siRNA can further be challenged in our model of Tagged-CycD1 reporter transgene, since we have shown that its targeting induces a tumor growth arrest but has no obvious incidence on the nude animal's health.

Material and Methods

Mice

Animal uses were performed in accordance with relevant guidelines and regulations. All experimental protocol were approved by the Regional Ethics committee (agreement number CEEA-LR-12070) and conducted according to approved procedures (Institute of Functional Genomics agreement number A 34-172-41, under F. Bienvenu agreement number A 34-513).

Ccnd1Ntag/Ntag and Ccnd1Ctag/Ctag mice have been described previously (Bienvenu et al. Nature 463, 374-378). Mice were bred at the Institute of Human Genetics animal care facility under standardized conditions with a 12 hours light/dark cycle, stable temperature (22±1° C.), controlled humidity (55±10%) and food and water ad libitum.

Genotyping of Cyclin D1-Tagged animals:

Genotyping of Ccnd1 Ntag/Ntag and Ccnd1Ctag/Ctag animals was done as previously (Bienvenu et al. Nature 463, 374-378).

In Vivo siRNA Delivery and Tumor Growth Analysis

siRNAs (Genecust or Sigma) were dissolved in nuclease-free water and stored at −20° C. until use as described before (Lehmann et al. PLoS ONE 9(2): e88797). The soluble/lipid formulation was prepared extemporaneously to transport siRNAs across the animal body. At a well-defined ratio according to manufacturer's instructions, the siRNA lipid monophasic micro-emulsion was obtained by short vortex mixing of the lipid constituents with the siRNA solution. The formulation was kept at room temperature and protected from light until use.

In vivo the formulation at 1 mg/mL of siRNA was administered by rectal route using a micropipette (Eline lite dispenser 12026368, Biohit) and adapted conical tips (Dispenser tips 792028, Biohit). A constant dosage-volume of 20 μl of siRNA formulation per delivery was used (1 mg/kg).

siRNA treatment for tumor progression analysis was done every day twice by injection in the anal mucosa of the mice in the morning and in the evening. Tumor sizes were measured and calculated from the following formula: tumor size=L×W2/2, where L and W represent the length and the width of the tumor mass respectively.

Where indicated, FLAG and HA siRNA delivery was done alternately to assess a synergic or additive effect. Alternating the FLAG or HA siRNA delivery did not provoke any substantial difference in tumor growth response compared to FLAG only or HA only. However, the inventors tested decreasing the siRNA dose (i.e 0.5 mg/mL instead of 1 mg/mL) but it induced a less dramatic tumor growth arrest on RAS-G12V/DNP53-driven tumors.

Allograft Animal Models

For allografts in vivo experiments in nude mice, TAG responding cells and control cells pairs were prepared by one experimentator (J.C. or B.M) who gave them to a second experimentator (L.K.L) who was blind to the nature of each cell line. The second experimentator (L.K.L.) implanted the cells subcutaneously. Then, L.K.L. or J.C. performed the siRNA delivery as described above, and measured the tumor sizes. For each experimental design, TAG positive responding cells were implanted on one flank of the mouse, while control cells were implanted on the contralateral flank of the same mouse. A minimum of 5 mice were used per experiment. For each siRNA preparation to be tested, that is empty vehicle, Scramble, Nat, Qia, Life, Flag or HA as mentioned, all the mice were treated every day at 9 AM and then at 5 PM. In the FIG. 5e of Extended Data, CycD1-null mice were treated with Nat-siRNA at 9 AM, then with Qia-siRNA at 12 AM and finally with Life-siRNA at 5 PM of the same day and for three consecutive days.

Where mentioned in the figures, treatment pause was applied and restarted later on when tumors reached larger sizes for experimental purposes.

In TAG-RAS-G12V experiment (FIG. 31, 33), TAG-siRNA targeting settings (twice per day) slows down tumor progression of TAG-RAS-G12V-driven tumors, but the treatment is not sufficient to induce a steady-state or regression of the tumor size. However, increasing the TAG-RNAi delivery frequency improves the tumor growth inhibition of this aggressive cancer model (not shown).

T286A transformed 3T3 cells: 2·10⁶ cells were used per site of subcutaneous implantation.

RAS-G12V/DNP53 transformed MEFs: 0.5·10⁶ cells were used per site of subcutaneous implantation.

H-RAS-G12V TAGOUT and H-RAS-G12V NoTAG transformed/Large T immortalized MEFs: 0.5·10⁶ cells were used per site of subcutaneous implantation.

Each cell type was resuspended in 150 μl of RPMI 1640 and inoculated into the subcutaneous flanks of 6 weeks old female athymic nude mice (Harlan).

Cells

Mouse Embryonic Fibroblast Cells

MEF cells were prepared as previously described.

Ccnd1−/−, Ccnd+/+ MEFs and wildtype 3T3 cells were kindly provided by P. Sicinski.

Cell Culture

MEFs derived cells were cultured in Dulbecco's Minimal Essential Medium (41966-029, Gibco), supplemented with 5% fetal bovine serum (Life technology) and 1000 U/ml of Penicillin-Streptomycin (P/S) (Gibco). All cells lines were incubated in a 37° C. incubator in an atmosphere of 5% C02 in air and maintained in sub-confluent culture conditions.

In Vitro siRNA Transfection

In-vitro siRNA delivery was done using Lipofectamine® RNAiMAX Transfection Reagent (Life Technologies) according to manufacturer's instructions. Cells to be transfected were seeded at 9 AM in the morning and transfected at 6 PM of the same day. The day after, cells were harvested at 9 AM or 6 PM for further biochemistry analysis.

Immunoblot

Immunoblots were performed as previously described (Bienvenu et al. Nature 463, 374-378). and with lysates obtained using HTRF lysis buffer (see below) supplemented with Protease Inhibitor Cocktail (S8830-20TAB). Antibodies used were HA (HA.11 Clone 16B12, Eurogentec, or Anti-HA EPITOPE TAG—600-401-384, Tebu-bio, or Hemagglutinin (HA) Rabbit Polyclonal Antibody, Life technologie), Cyclin D1 (sc-450, Santa cruz, or MS-210-PABX (AB1), Fisher scientific or RB-010-PABX (AB3), Fisher scientific), Actin (ab6276, Abcam), Tubulin (T9026, Sigma-Aldrich), Flag (F7425, Sigma-Aldrich), Ras (BD610002, BD Biosciences), Cyclin E1 (sc-481, Santa Cruz), CDK4 (sc-260, Santa Cruz), Cyclin D2 (MS-221-PABX (AB4), Fisher scientific), Cyclin D3 (MS-215-PABX (AB1), Fisher scientific). As secondary antibodies, peroxidase-conjugated IgG (Cell signaling) was used, followed by enhanced chemiluminescence detection (Millipore) and revealed with ChemiDoc™ XRS+ System (Biorad).

Tandem-HTRF

Cells in culture were washed with 1×PBS at 37° C. and then lysed in HTRF lysis buffer (Tris 10 mM, EDTA 1 mM, 0.05% NP-40). After centrifugation at 16000 g for 10 minutes, samples normalization were performed by adjusting total DNA content (nanodrop, Thermo Scientific) to 50 ng/μL. In each control experiment wild type cyclin D1 (or Cyclin D1-null) samples were used as negative control of noise signal (control 1). In addition, samples to be analyzed were incubated with donor antibody only in parallel (control 2). Comparison of both controls was performed for each Tandem-HTRF measure and gives identical background results19.

Tandem-HTRF detection of Cyclin D1 was performed with donor and acceptor antibody mixes according to manufacturer's instructions (Cisbio Bioassays—0.4 nM for the donor and 3 nM for the acceptor) within the linear range of HTRF signal (inside the linearity window of antibodies), to avoid high level saturation and keep low noise level. Donor antibodies were labeled with Europium (Eu) or Terbium (Tb) Cryptate fluorophore, and acceptor antibodies were labeled with XL665 fluorophore, or d2. List of antibodies can be found in Extended Data supplemental information.

Three independent samples were processed separately (biological triplicate) for Tandem-HTRF reaction. Each Tandem-HTRF sample being performed in technical triplicates as well.

The labeling of antibodies was made by the manufacturer Cisbio bioassays (to be contacted for more information).

For Tandem-HTRF measure, antibodies mix were diluted in q.s.p 5 μl of 0.2×PBS and added to 5 μl of sample per well of a Greiner black 384-well plate. After shaking and centrifugation (600 g for 1 minute), samples were kept at 4° C. overnight, protected from light.

HTRF was acquired by a PHERAstar FS microplate reader (BMG Labtech) as follows: after excitation with a laser at 337 nm (40 flashes per well), fluorescence emissions were monitored both at 620 nm (Lumi4-Tb emission) and at 665 nm (XL665 and d2 emission). A 400-μs integration time was used after a 60-μs delay to remove the short-lived fluorescence background from the specific signal.

The HTRF intensity was calculated using the following formula and is expressed as arbitrary units:

HTRF(intensity)={(ratio 665/620)sample}×10\hat{ }4−{(ratio 665/620)background}×10\hat{ }4

The background signal corresponds to cell lysates labeled with the Lumi4-Tb alone or control cell lysates devoid of the bait (wildtype cells). For each HTRF measure, the mean of technical replicates were used. Tandem-HTRF results outlined in the figures are the average of three biological independent experiments +/− standard deviation unless mentioned otherwise.

Retroviral Constructs

Plasmids:

All Cyclin D1 or RAS genetic constructs were inserted into BamH1-EcoR1 restriction sites of retro-viral vector pBABE-Puro or pBABE-hygro kindly provided by P. Sicinski, or MSCV retro-viral vector kindly provided by O. Ayrault. Large T encoding plasmid was kindly provided by L. Fajas, Ras-G12V/DNP53 plasmid (pL56-Ras) was kindly provided by L. LeCam. mCherry cDNA (CMV-mCherry) was kindly provided by V. Homburger and inserted into SnaB1-NotI restriction site of MSCV vector. Inserts sequences are listed in supplementary information.

Generation of Cyclin D1 Rescue or H-RAS Inserts

All retroviral constructs used were manipulated according to security measures and approved by the Institute of Functional Genomics.

cDNA Inserts of mouse origin (Cyclin D1 and Hras) were generated by RT-PCR using cDNA template from Ccnd1Ntag/Ctag E.13.5 embryonic head derived from C57BL/6j×129Sv mixed genetic background. The PCR products were inserted into retro-viral vectors and verified by sequencing after bacterial amplification.

Mutagenesis

T286A-CycD1 mutagenesis was performed using GeneArt@ Site-Directed Mutagenesis System (LifeTechnologies) according to manufacturer's recommendations.

Mutagenesis Primers are listed in Supplementary information.

G12V-Kras and WT-Kras Oligomers

Oligos were ordered at IDT-DNA and inserted into EcoR1-BgIII restriction sites of MSCV-Ntag-CycD1-Puro vector. An additional NdeI restriction site was used for cloning verification before sequencing of the resulting plasmid construct. Sequence of the oligos can be found in supplementary information below.

V600E-Braf and WT-Braf Oligomers

Oligos were ordered at IDT-DNA and inserted into BgIII-NotI restriction sites of MSCV-CycD1-Puro vector. An additional MfeI restriction site was used for cloning verification before sequencing of the resulting plasmid construct. Sequence of the oligos can be found in supplementary information below.

Stable Cell Lines Generation

Cells obtained by retroviral infection were done as described (Bienvenu et al. Nature 463, 374-378). Briefly, the day before transfection, Plat-E cells were seeded in 10 cm dishes at 50% confluence in DMEM (Gibco) supplemented with 10% Fetal Bovine Serum (Life technology).

Murine ecotrope retroviruses were produced by jetPEI transfection of Plat-E cells with 3 μg of pBabe-puro or MSCV-puro transfer vector or empty control vector (no resistance). 48 h after transfection, viral supernatant was harvested, filtered (0.45 um), supplemented with 8 μg/ml polybrene (H9268, Sigma) and used to infect recipient proliferating cells. 72 h after infection, medium of recipient cells was replaced and cells were selected for several days with 2 μg/ml of puromycin or 150 μg/ml of hygromycin, until all control cells exposed to empty virus are dead.

RT-qPCR

RNA Preparation

Total RNA was prepared using Trizol (Invitrogen) according to the manufacturer's instruction. Purified RNA was treated with the DNase I from the DNA-free™ kit (Ambion) according to manufacturer's instructions.

Reverse Transcription

1 μg of total RNA was reverse transcribed using 200 U M-MLV reverse transcriptase (Invitrogen) in the presence of 2.5 μM random hexamers, 0.5 mM dNTP, 10 mM DTT and 40 U of RNAse inhibitor (Invitrogen).

Real-Time PCR to Semi-Quantify Cyclin D1 mRNA

Four ng of the RT resulting cDNAs were used as template for real time PCR using LightCycler®480 Real-Time PCR System (Roche Applied Science) with the LightCycler® 480 SYBR Green I Master (Roche Applied Science). The sequences of all the primers used are listed in Extended Data. The PCR reaction was performed in 5 μl in the presence of 300 nM specific primers. Thermal cycling parameters were 10 min at 95° C., followed by 45 cycles of 95° C. for 15 s and 60° C. for 30 sec. At the end of the PCR, melting curve analyses of amplification products were carried out to confirm that only one product was amplified. The level of expression of each gene “X” was normalized to the geometric mean of the expression levels of the selected reference genes, R1 to R3, in the same PCR plate according to the formula:

Reference genes were selected among eight commonly used according to the GeNorm procedure (http://medgen.ugent.be/˜jvdesomp/genorm/). Reference genes tested in this study were B2m (beta-2 microglobulin), Gapdh (glyceraldehyde-3-phosphate dehydrogenase), Mrpl32 (mitochondrial 39S ribosomal protein L32), Tbp (TFIID) (TATA box binding protein), Tubb2b (Tubulin beta2b), Trfr1 (transferrin receptor-1), all listed in Extended Data.

RNA-Sequencing

RNA Libraries Generation

RNA-Seq libraries were constructed with the Truseq stranded mRNA sample preparation (Low throughput protocol) kit from Illumina.

Poly-A Based mRNA Enrichment

One microgram of total RNA was used for the construction of the libraries,

The first step in the workflow involves purifying the poly-A containing mRNA molecules using poly-T oligo attached magnetic beads. Following purification, the mRNA is fragmented into small pieces using divalent cations under elevated temperature. The cleaved RNA fragments are copied into first strand cDNA using SuperScript II reverse transcriptase, Actinomycine D and random hexamer primers. The Second strand cDNA was synthesized by replacing dTTP with dUTP. These cDNA fragments then have the addition of a single ‘A’ base and subsequent ligation of the adapter. The products are then purified and enriched with 15 cycles of PCR. The final cDNA libraries were validated with a DNA 1000 Labchip on a Bioanalyzer (Agilent) and quantified with a KAPA qPCR kit.

For each sequencing lane of a flowcell V3, four libraries were pooled in equal proportions, denatured with NaOH and diluted to 7.5 μM before clustering. Cluster formation, primer hybridisation and single end-read 50 cycles sequencing were performed on cBot and HiSeq2000 (Illumina, San Diego, Calif.) respectively.

RNA-Sequencing Statistical Analysis

Image analysis and base calling were performed using the HiSeq Control Software and Real-Time Analysis component. Demultiplexing was performed using Illumina's sequencing analysis software (CASAVA 1.8.2). The quality of the data was assessed using FastQC from the Babraham Institute and the Illumina software SAV (Sequence Analysis Viewer). Potential contaminants were investigated with the FastQ Screen software from the Babraham Institute.

RNA-seq reads were aligned to the mouse genome (UCSC mm10) with a set of gene model annotations (genes.gtf downloaded from UCSC on May 23 2014; GeneIDs come from the NCBI: gene2refseq.gz downloaded on Sep. 24 2015), using the splice junction mapper TopHat 2.0.1333 (with bowtie 2.2.334). Final read alignments having more than 3 mismatches were discarded. Gene counting was performed using HTSeq-count 0.6.1 μl (union mode)35. Since data come from a strand-specific assay, the read has to be mapped to the opposite strand of the gene. Before statistical analysis, genes with less than 20 reads (cumulating all the analysed samples) were filtered out.

DESeq2

Differentially expressed genes were identified using the Bioconductor36 package DESeq2 1.4.537. Data were normalized using the DESeq2 normalization method. Genes with adjusted p-value less than 5% (according to the FDR method from Benjamini-Hochberg) were declared differentially expressed. Generalized linear models was used to take into account paired samples.

Statistical Analysis

Data and statistical methods are expressed as outlined in figure legends. The means of two groups were compared using two-tailed paired or unpaired Student's t test.

Supplementary Information

Primers Used for T286A CycD1 Mutagenesis

Forward primer:
SEQ ID NO: 52
GGTCTGGCCTGCGCGCCCACCGACGTG-
Reverse primer:
SEQ ID NO: 53
CACGTCGGTGGGCGCGCAGGCCAGACC-

RT-qPCR Primers

| | | | | SEQ |
|---|---|---|---|---|
| | | | | ID |
| Gène | SeqRef | Forward | Forward Sequence | NO: |
| B2μg (beta2 | NM_ | B2m- | TATGCTATCCAGAAAA | 54 |
| microglobulin) | 009735 | F | CCCCTCAA | |
| GAPDH | NM_ | Gapdh- | GGAGCGAGACCCCACT | 55 |
| glyceraldehyde- | 008084 | F | AACA | |
| 3-phosphate | | | | |
| dehydrogenase | | | | |
| Trfr1 | NM_ | Trfr1- | AGACCTTGCACTCTTT | 56 |
| (transferrin | 011638 | F | GGACATG | |
| receptor-1) | | | | |
| Mrpl32 | NM_ | Mrpl32- | AGGTGCTGGGAGCTGC | 57 |
| (mitochondrial | 029271 | F | TACA | |
| 39S ribosomal | | | | |
| protein L32) | | | | |
| Tbp (TFIID) | NM_ | Tbp2a- | ATCGAGTCCGGTAGCC | 58 |
| TATA box | 013684 | F | GGTG | |
| binding | | | | |
| protein | | | | |
| TUBULIN, | NM_ | Tubb2b- | CTTAGTGAACTTCTGT | 59 |
| BETA-2B | 023716 | F | TGTCCTCCA | |
| Cyclin D1 | NM_ | mCcnd1- | AGGAGCAGAAGTGCGA | 60 |
| [Mus | 007631 | F | AGAG | |
| musculus] | | | | |
| mCherry | | mCherry- | CCTGTCCCCTCAGTTC | 61 |
| | | F | ATGT | |
| Gène | SeqRef | Reverse | Reverse Sequence | |
| B2μg (beta2 | NM_ | B2m- | GTATGTTCGGCTTCCC | 62 |
| microglubin) | 009735 | R | ATTCTC | |
| GAPDH | NM_ | Gapdh- | ACATACTCAGCACCGG | 62 |
| glyceraldehyde- | 008084 | R | CCTC | |
| 3-phosphate | | | | |
| dehydrogenase | | | | |
| Gus (beta- | NM_ | Gus2- | GCCAACGGAGCAGGTT | 64 |
| glucuronidase) | 010368 | R | GA | |
| Trfr1 | NM_ | Trfr1- | GGTGTGTATGGATCAC | 65 |
| (transferrin | 011638 | R | CAGTTCCTA | |
| receptor-1) | | | | |
| Mrpl32 | NM_ | Mrpl32- | AAAGCGACTCCAGCTC | 66 |
| (mitochondrial | 029271 | R | TGCT | |
| 39S ribosomal | | | | |
| protein L32) | | | | |
| Tbp (TFIID) | NM_ | Tbp2a- | GAAACCTAGCCAAACC | 67 |
| TATA box | 013684 | R | GCC | |
| binding | | | | |
| protein | | | | |
| TUBULIN, | NM_ | Tubb2b- | AGGCAAACTGAGCACC | 68 |
| BETA-2B | 023716 | R | ATAATTTACAAA | |
| Cyclin D1 | NM_ | mCcnd1- | CACAACTTCTCGGCAG | 69 |
| [Mus | 007631 | R | TCAA | |
| musculus] | | | | |
| mCherry | | mCherry- | CCCATGGTCTTCTTCT | 70 |
| | | F | GCAT | |

TABLE 1

| siRNA | Active anti-sens | SEQ ID | | SEQ ID |
|---|---|---|---|---|
| name | sequence 5′-3′ | NO: | Non-active Sense sequence 5′-3′ | NO: |
| Scramble | AAUUCUCCGAAC | 71 | ACGUGACACGUUCGGAGAAtt | 121 |
| | GUGUCACGU | | | |
| HA | UAGUCGGGCACG | 72 | CCUACGACGUGCCCGACUAtt | 122 |
| | UCGUAGGGG | | | |
| FLAG | GUCAUCGUCGUC | 73 | CUACAAGGACGACGAUGACtt | 123 |
| | CUUGUAGUC | | | |
| FLAGC | CGACUUGUCAUC | 74 | GGACGACGAUGACAAGUCGtt | 124 |
| | GUCGUCCUU | | | |
| FLAGN | GAGCUUGUCAUC | 75 | GGACGACGAUGACAAGCUCtt | 125 |
| | GUCGUCCUU | | | |
| Nat | CCACAGAUGUGA | 76 | AAAUGAACUUCACAUCUGUG | 126 |
| | AGUUCAUUU | | Gtt | |
| Qia | AACACCAGCUCC | 77 | CGCAGCACAGGAGCUGGUGU | 127 |
| | UGUGCUGCG | | Utt | |
| Life | CAGGAACAGAUU | 78 | AAGGGCUUCAAUCUGUUCCU | 128 |
| | GAAGCCCUU | | Gtt | |
| V5#1 | cggguucggaaucggu | 79 | caaaccgauuccgaacccgTT | 129 |
| | uugcc | | | |
| V5#2 | gcggguucggaaucgg | 80 | aaaccgauuccgaacccgcTT | 130 |
| | uuugc | | | |
| V5#4 | cagcggguucggaauc | 81 | accgauuccgaacccgcugTT | 131 |
| | gguuu | | | |
| V5#5 | gcagcggguucggaau | 82 | ccgauuccgaacccgcugcTT | 132 |
| | cgguu | | | |
| V5#6 | agcagcggguucggaa | 83 | cgauuccgaacccgcugcuTT | 133 |
| | ucggu | | | |
| V5#7 | cagcagcggguucgga | 84 | gauuccgaacccgcugcugTT | 134 |
| | aucgg | | | |
| V5#8 | ccagcagcggguucgg | 85 | auuccgaacccgcugcuggTT | 135 |
| | aaucg | | | |
| V5#9 | cccagcagcggguucg | 86 | uuccgaacccgcugcugggTT | 136 |
| | gaauc | | | |
| V5#10 | gcccagcagcggguuc | 87 | uccgaacccgcugcugggcTT | 137 |
| | ggaau | | | |
| V5#11 | ggcccagcagcggguu | 88 | ccgaacccgcugcugggccTT | 138 |
| | cggaa | | | |
| V5#12 | aggcccagcagcgggu | 89 | cgaacccgcugcugggccuTT | 139 |
| | ucgga | | | |
| V5#13 | caggcccagcagcggg | 90 | gaacccgcugcugggccugTT | 140 |
| | uucgg | | | |
| V5#14 | ccaggcccagcagcgg | 91 | aacccgcugcugggccuggTT | 141 |
| | guucg | | | |
| V5#15 | uccaggcccagcagcg | 92 | acccgcugcugggccuggaTT | 142 |
| | gguuc | | | |
| V5#16 | auccaggcccagcagc | 93 | cccgcugcugggccuggauTT | 143 |
| | ggguu | | | |
| V5#17 | uauccaggcccagcag | 94 | ccgcugcugggccuggauaTT | 144 |
| | cgggu | | | |
| V5#18 | cuauccaggcccagca | 95 | cgcugcugggccuggauagTT | 145 |
| | gcggg | | | |
| V5#19 | gcuauccaggcccagc | 96 | gcugcugggccuggauagcTT | 146 |
| | agcgg | | | |
| V5#20 | ugcuauccaggcccag | 97 | cugcugggccuggauagcaTT | 147 |
| | cagcg | | | |
| V5#21 | gugcuauccaggccca | 98 | ugcugggccuggauagcacTT | 148 |
| | gcagc | | | |
| V5#22 | ggugcuauccaggccc | 99 | gcugggccuggauagcaccTT | 149 |
| | agcag | | | |
| G12V- | ACAGCUCCAACU | 100 | UUGUGGUAGUUGGAGCUGUtt | 150 |
| Ras- | ACCACAAGC | | | |
| Tag#1 | | | | |
| G12V- | AACAGCUCCAAC | 101 | UGUGGUAGUUGGAGCUGUUtt | 151 |
| Ras- | UACCACAAG | | | |
| Tag#2 | | | | |
| G12V- | CAACAGCUCCAA | 102 | GUGGUAGUUGGAGCUGUUGtt | 152 |
| Ras- | CUACCACAA | | | |
| Rag#3 | | | | |
| G12V- | CCAACAGCUCCA | 103 | UGGUAGUUGGAGCUGUUGGtt | 153 |
| Ras- | ACUACCACA | | | |
| Tag#4 | | | | |
| G12V- | GCCAACAGCUCC | 104 | GGUAGUUGGAGCUGUUGGCtt | 154 |
| Ras- | AACUACCAC | | | |
| Tag#5 | | | | |
| G12V- | CGCCAACAGCUC | 105 | GUAGUUGGAGCUGUUGGCGtt | 155 |
| Ras- | CAACUACCA | | | |
| Tag#6 | | | | |
| G12V- | ACGCCAACAGCU | 106 | UAGUUGGAGCUGUUGGCGUtt | 156 |
| Ras- | CCAACUACC | | | |
| Tag#7 | | | | |
| G12V- | UACGCCAACAGC | 107 | AGUUGGAGCUGUUGGCGUAtt | 157 |
| Ras- | UCCAACUAC | | | |
| Tag#8 | | | | |
| G12V- | CUACGCCAACAG | 108 | GUUGGAGCUGUUGGCGUAGtt | 158 |
| Ras- | CUCCAACUA | | | |
| Tag#9 | | | | |
| G12V- | CCUACGCCAACA | 109 | UUGGAGCUGUUGGCGUAGGtt | 159 |
| Ras- | GCUCCAACU | | | |
| Tag#10 | | | | |
| G12V- | GCCUACGCCAAC | 110 | UGGAGCUGUUGGCGUAGGCtt | 160 |
| Ras- | AGCUCCAAC | | | |
| Tag#11 | | | | |
| G12V- | UGCCUACGCCAA | 111 | GGAGCUGUUGGCGUAGGCAtt | 161 |
| Ras- | CAGCUCCAA | | | |
| Tag#12 | | | | |
| G12V- | UUGCCUACGCCA | 112 | GAGCUGUUGGCGUAGGCAAtt | 162 |
| Ras- | ACAGCUCCA | | | |
| Tag#13 | | | | |
| G12V- | CUUGCCUACGCC | 113 | AGCUGUUGGCGUAGGCAAGtt | 163 |
| Ras- | AACAGCUCC | | | |
| Tag#14 | | | | |
| G12V- | UCUUGCCUACGC | 114 | GCUGUUGGCGUAGGCAAGAtt | 164 |
| Ras- | CAACAGCUC | | | |
| Tag#15 | | | | |
| G12V- | CUCUUGCCUACG | 115 | CUGUUGGCGUAGGCAAGAGtt | 165 |
| Ras- | CCAACAGCU | | | |
| Tag#16 | | | | |
| G12V- | ACUCUUGCCUAC | 116 | UGUUGGCGUAGGCAAGAGUtt | 166 |
| Ras- | GCCAACAGC | | | |
| Tag#17 | | | | |
| G12V- | CACUCUUGCCUA | 117 | GUUGGCGUAGGCAAGAGUGtt | 167 |
| Ras- | CGCCAACAG | | | |
| Tag#18 | | | | |
| G12V- | GCACUCUUGCCU | 118 | UUGGCGUAGGCAAGAGUGCtt | 168 |
| Ras- | ACGCCAACA | | | |
| Tag#19 | | | | |
| G12V- | GGCACUCUUGCC | 119 | UGGCGUAGGCAAGAGUGCCtt | 169 |
| Ras- | UACGCCAAC | | | |
| Tag#20 | | | | |
| G12V- | UGGCACUCUUGC | 120 | GGCGUAGGCAAGAGUGCCAtt | 170 |
| Ras- | CUACGCCAA | | | |
| Tag#21 | | | | |
| V600E- | UCUGUAGCUAGA | 183 | AUUUUGGUCUAGCUACAGatt | 204 |
| Raf- | CCAAAAUCA | | | |
| Tag#1 | | | | |
| V600E- | CUCUGUAGCUAG | 184 | UUUUGGUCUAGCUACAGaGtt | 205 |
| Raf- | ACCAAAAUC | | | |
| Tag#2 | | | | |
| V600E- | UCUCUGUAGCUA | 185 | UUUGGUCUAGCUACAGaGAtt | 206 |
| Raf- | GACCAAAAU | | | |
| Tag#3 | | | | |
| V600E- | UUCUCUGUAGCU | 186 | UUGGUCUAGCUACAGaGAAtt | 207 |
| Raf- | AGACCAAAA | | | |
| Tag#4 | | | | |
| V600E- | UUUCUCUGUAGC | 187 | UGGUCUAGCUACAGaGAAAtt | 208 |
| Raf- | UAGACCAAA | | | |
| Tag#5 | | | | |
| V600E- | AUUUCUCUGUAG | 188 | GGUCUAGCUACAGaGAAAUtt | 209 |
| Raf- | CUAGACCAA | | | |
| Tag#6 | | | | |
| V600E- | GAUUUCUCUGUA | 189 | GUCUAGCUACAGaGAAAUCtt | 210 |
| Raf- | GCUAGACCA | | | |
| Tag#7 | | | | |
| V600E- | AGAUUUCUCUGU | 190 | UCUAGCUACAGaGAAAUCUtt | 211 |
| Raf- | AGCUAGACC | | | |
| Tag#8 | | | | |
| V600E- | GAGAUUUCUCUG | 191 | CUAGCUACAGaGAAAUCUCtt | 212 |
| Raf- | UAGCUAGAC | | | |
| Tag#9 | | | | |
| V600E- | CGAGAUUUCUCU | 192 | UAGCUACAGaGAAAUCUCGtt | 213 |
| Raf- | GUAGCUAGA | | | |
| Tag#10 | | | | |
| V600E- | UCGAGAUUUCUC | 193 | AGCUACAGaGAAAUCUCGAtt | 214 |
| Raf- | UGUAGCUAG | | | |
| Tag#11 | | | | |
| V600E- | AUCGAGAUUUCU | 194 | GCUACAGaGAAAUCUCGAUtt | 215 |
| Raf- | CUGUAGCUA | | | |
| Tag#12 | | | | |
| V600E- | CAUCGAGAUUUC | 195 | CUACAGaGAAAUCUCGAUGtt | 216 |
| Raf- | UCUGUAGCU | | | |
| Tag#13 | | | | |
| V600E- | CCAUCGAGAUUU | 196 | UACAGaGAAAUCUCGAUGGtt | 217 |
| Raf- | CUCUGUAGC | | | |
| Rag#14 | | | | |
| V600E- | UCCAUCGAGAUU | 197 | ACAGaGAAAUCUCGAUGGAtt | 218 |
| Raf- | UCUCUGUAG | | | |
| Tag#15 | | | | |
| V600E- | CUCCAUCGAGAU | 198 | CAGaGAAAUCUCGAUGGAGtt | 219 |
| Raf- | UUCUCUGUA | | | |
| Tag#16 | | | | |
| V600E- | ACUCCAUCGAGA | 199 | AGaGAAAUCUCGAUGGAGUtt | 220 |
| Raf- | UUUCUCUGU | | | |
| Tag#17 | | | | |
| V600E- | CACUCCAUCGAG | 200 | GaGAAAUCUCGAUGGAGUGtt | 221 |
| Raf- | AUUUCUCUG | | | |
| Tag#18 | | | | |
| V600E- | CCACUCCAUCGA | 201 | aGAAAUCUCGAUGGAGUGGtt | 222 |
| Raf- | GAUUUCUCU | | | |
| Tag#19 | | | | |
| V600E- | CCCACUCCAUCG | 202 | GAAAUCUCGAUGGAGUGGGtt | 223 |
| Raf- | AGAUUUCUC | | | |
| Tag#20 | | | | |
| V600E- | ACCCACUCCAUC | 203 | AAAUCUCGAUGGAGUGGGUtt | 224 |
| Raf- | GAGAUUUCU | | | |
| Tag#21 | | | | |

Oligos Used for G12VTAG Specific siRNA Screening

EcoR1 G12V-Endotag Bglll TOP
(SEQ ID NO: 171)
AATTCCATATGCTTGTGGTAGTTGGAGCTGt
TGGCGTAGGCAAGAGTGCCA,
EcoR1 G12V-Endotag Bglll Bottom
(SEQ ID NO: 172)
gatcTGGCACTCTTGCCTACGCCAaCAGCTC
CAACTACCACAAGCATATGg,
EcoR1 WT-Endotag Bglll TOP
(SEQ ID NO: 173)
AATTCCATATGCTTGTGGTAGTTGGAGCTGg
TGGCGTAGGCAAGAGTGCCA,
EcoR1 WT-Endotag Bglll Bottom
(SEQ ID NO: 174)
gatcTGGCACTCTTGCCTACGCCAcCAGCTC
CAACTACCACAAGCATATGg,

Oligos Used for V600E-TAG Specific siRNA Screening

Bam-BRAFwtTAG-Not-TOP
(SEQ ID NO: 225)
GATCCCAATTGTAGTTAGTTTAGACCGGTTGATTTTGGTCTAGCTAC
AGtGAAATCTCGATGGAGTGGGTACGCGTAGATCTTATTTGC
Bam-BRAFwtTAG-Not-Bottom
(SEQ ID NO: 226)
ggccGCAAATAAGATCTACGCGTACCCACTCCATCGAGATTTCaCTG
TAGCTAGACCAAAATCAACCGGTCTAAACTAACTACAATTGG
Bam-BRAFv600eTAG-Not-TOP
(SEQ ID NO: 227)
GATCCCAATTGTAGTTAGTTTAGACCGGTTGATTTTGGTCTAGCTAC
AGaGAAATCTCGATGGAGTGGGTACGCGTAGATCTTATTTGC
Bam-BRAFv600eTAG-Not-Bottom
(SEQ ID NO: 228)
ggccGCAAATAAGATCTACGCGTACCCACTCCATCGAGATTTCtCTG
TAGCTAGACCAAAATCAACCGGTCTAAACTAACTACAATTGG

List of Tandem-HTRF Antibodies
HA-XL, 610HAXLB, Flag-Tb, 61FG2TLB, and MYC-Eu, 61MYCKLA, Cisbio
V5-d2, 64CUSDAYE, Cisbio (custom labelling of MA5-15253 (V5), Perbio)
AB3-d2 64CUSDAZE, Cisbio (custom labelling of RB-010-PABX (AB3), Fisher scientific)
AB1-d2 64CUSDAZE, Cisbio (custom labelling of MS-210-PABX (AB1), Fisher scientific)
SC-450-d2 64CUSDAZE, Cisbio (custom labelling of SC-450, Santa Cruz)

Analysis of sequence homology between siRNAs and mouse CycD1, CycD2, CycD3 and CycE1 cDNA:

Potential off-target hybridization sites: 5
Flag siRNA sequence:
(SEQ ID NO: 175)
GACTACAAGGACGACGATGAC.
Potential off-target hybridization sites: 7
HA siRNA sequence:
(SEQ ID NO: 176)
CCCCTACGACGTGCCCGACTA.
Potential off-target hybridization sites: 23
Nat siRNA sequence:
(SEQ ID NO: 177)
CCACAGATGTGAAGTTCATTT.
Potential off-target hybridization sites: 18
Qiagen siRNA sequence:
(SEQ ID NO: 178)
AACACCAGCTCCTGTGCTGCG
Potential off-target hybridization sites: 16
Life siRNA sequence:
(SEQ ID NO: 179)
CAGGAACAGATTGAAGCCCTT.

1. A nucleic acid molecule comprising:
a first region comprising a nucleic acid sequence coding for the protein Cyclin D1, also called CCND1, said first region being controlled by means allowing the expression of said protein, and
at least one second region, said second region comprising essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide.

2. The nucleic acid molecule according to claim 1, in which said first region comprise one of the following sequences coding for said CCND1 protein: SEQ ID NO: 1 or SEQ ID NO: 2.

3. The nucleic acid molecule according to claim 1, wherein said means allowing expression of said CCND1 protein are means allowing translation initiation by ribosomes.

4. The nucleic acid molecule according to claim 1, wherein said first region comprises or consists essentially of one of the following sequences: SEQ ID NO: 5 to SEQ ID NO: 10 or SEQ ID NO: 30 to SEQ ID NO: 35.

5. The nucleic acid molecule according to claim 1, wherein said first region is located in a 5′ position of said second region.

6. The nucleic acid molecule according to claim 1, wherein said first region is located in a 3′ position of said second region.

7. The nucleic acid molecule according to claim 1, wherein said second region is genetically isolated from said first region by at least one sequence of end of translation.

8. The nucleic acid molecule according to claim 1, wherein said nucleic acid molecule comprises one of the sequences as set forth in SEQ ID NO: 11, SEQ ID NO: 15, SEQ ID NO: 19, SEQ ID NO: 36, SEQ ID NO: 40, and SEQ ID NO: 44.

9. A cell comprising at least one copy of the nucleic acid molecule as defined in claim 1.

10. A genetically modified non-human animal, comprising at least one cell as defined in claim 9.

11. A transgenic non-human animal having a modified endogenous CCND1 coding gene, said gene being modified
either by the insertion, directly upstream of the translation initiation sequence containing the first ATG of the first exon of said CCND1 gene, a sequence consisting of at least one second region,
or by the insertion, directly downstream of the translation termination sequence containing the stop codon of the last exon of said CCND1 gene, a sequence consisting of at least one second region,
wherein said second region comprises essentially a sequence from 14 to 59 nucleic acids, said second region corresponding to a transcribed region of a gene, said transcribed region of a gene containing at least a genetic modification compared to the same transcribed region of the corresponding wild-type version of said gene, said second region being genetically isolated from the means allowing the expression of said protein such that said transcribed region of a gene is not translated into a peptide.

12. A subset of nucleic acid molecules, comprising
A first nucleic acid molecule as defined in claim 1, and
A second nucleic acid molecule, said second nucleic acid molecule comprising,
i. The same first region compared to the first region of said first nucleic acid molecule, and possibly
ii. At least a second region, said second region comprising essentially a sequence from 14 to 59 nucleic acids, said second region corresponding to a transcribed region of a gene, said second region comprising the wild-type version of said gene compared to the second region of said first nucleic acid molecule, said second region being genetically isolated from the means allowing the expression of said protein such that said second region is not translated into a peptide.

13. A set of nucleic acid molecules, comprising:
i. A subset according to claim 12,
ii. A third nucleic acid molecule, said second nucleic acid molecule comprising,
A first region comprising a nucleic acid sequence coding for reporter protein, said first region being controlled by means allowing translation of said reporter protein, and
A second region corresponding to the second region found in the first nucleic acid molecule, and
iii. A fourth nucleic acid molecule comprising,
A first region corresponding to the first region of said third nucleic acid molecule,
and possibly
At least a second region, said second region comprising essentially a sequence from 14 to 59 nucleic acids, said second region corresponding to a transcribed region of a gene, said second region comprising the wild-type version of said gene compared to the second region of said first or third nucleic acid molecule, said second region being genetically isolated from the means allowing the expression of said protein such that said second region is not translated into a peptide.

14. (canceled)

15. A method for screening of small interfering nucleic acid molecules comprising a step of contacting a tumoral cell containing
at least a nucleic acid molecule according to claim 1
with small interfering nucleic acid molecules, and
a step of evaluating said tumoral cell homeostasis.

16. A method for in vitro identifying the tumoral effect of nucleic acid sequence containing a genetic modification compared to its wild type counterpart, said method comprising a step of contacting a tumoral cell containing a set according to claim 13 with small interfering nucleic acid molecules.

17. A method for screening small interfering nucleic acid molecules allowing a tumor regression comprising the steps of:
injecting tumoral cells comprising at least a nucleic acid molecule according to claim 1 into an immunosuppressed non-human animal, possible an immunosuppressed mouse or rat, in order to allow a tumor growth,
injecting into the growing tumor a small interfering nucleic acid molecule at least complementary to the second region contained in said at least a nucleic acid molecule, and selecting the small interfering nucleic acid molecule allowing a tumor regression.

18. The cell according to claim 9, wherein said cell is a tumoral cell.

19. A method for screening of small interfering nucleic acid molecules comprising a step of contacting a tumoral cell containing a subset of nucleic acid molecules according to claim 12 with small interfering nucleic acid molecules, and a step of evaluating said tumoral cell homeostasis.

20. A method for screening of small interfering nucleic acid molecules comprising a step of contacting a tumoral cell containing a set of nucleic acid molecules according to claim 13 with small interfering nucleic acid molecules, and a step of evaluating said tumoral cell homeostasis.
